(19) United States
(12) Patent Application Publication
(10) Pub. No.: US 2015/0359849 A1
(43) Pub. Date: Dec. 17, 2015

(54) METHODS OF INCREASING NEURONAL CONNECTIVITY AND/OR TREATING A NEURODEGENERATIVE CONDITION

(71) Applicant: PRESIDENT AND FELLOWS OF HARVARD COLLEGE, Cambridge, MA (US)

(72) Inventors: Michael Eldon GREENBERG, Brookline, MA (US); Bulent ATAMAN, Cambridge, MA (US)

(21) Appl. No.: 14/764,082

(22) PCT Filed: Jan. 31, 2014
(86) PCT No.: PCT/US14/14169

§ 371 Date: Jul. 28, 2015

Related U.S. Application Data
(60) Provisional application No. 61/759,244, filed on Jan. 31, 2013.

(51) Int. Cl. A61K 38/17; C12Q 1/68

(52) U.S. Cl. CPC A61K 38/1709; C12Q 1/6883; C12Q 2600/158

(57) ABSTRACT

The inventions provided herein relate to methods, and compositions for increasing neuronal connectivity, neuronal survival and/or axonal growth of a population of neural cells in vitro, ex vivo or in vivo. For in vivo applications, in some embodiments, the methods and compositions described herein can be used to treat cognitive, motor and/or sensory function impairment and/or neurodegeneration in a subject or particularly a human subject {e.g., a human subject who is diagnosed as having, or having a risk, for a neurodegenerative and/or neurological condition such as Alzheimer's disease). Methods for determining a risk for a neurodegenerative condition or disorder, e.g., Alzheimer's disease, in a subject {e.g., a human subject) are also provided herein.

### CROSS-REFERENCE TO RELATED APPLICATIONS

This application claims benefit under 35 U.S.C. §119(e) of the U.S. Provisional Application No. 61/759,244 filed Jan. 31, 2013, the content of which is incorporated herein by reference in its entirety.

### GOVERNMENT SUPPORT

This invention was made with government support under Grant No. 1 RC2MH089952 awarded by the National Institutes of Health. The government has certain rights in the invention.

### TECHNICAL FIELD OF THE DISCLOSURE

The inventions provided herein generally relate to methods and compositions for increasing neuronal connectivity and/or neuronal survival of a population of neural cells. In one aspect, the inventions provided herein relate to methods and compositions for treating a neurodegenerative and/or neurological condition or disorder, e.g., Alzheimer's disease, and/or diseases or conditions where axonal growth and/or regeneration is desired to be promoted. Methods for determining a risk for cognitive impairment and/or a neurodegenerative condition or disorder, e.g., Alzheimer's disease, in a subject are also provided herein.

### BACKGROUND

Neurodegenerative and/or neurological disorders affect millions of individuals. For example, Alzheimer's disease (AD) is a devastating neurodegenerative disorder which is clinically characterized by deterioration of memory and cognitive function, progressive impairment of daily living activities, and several neuropsychiatric symptoms. Cummings, J. L., 351 N Engl J Med. 56 (2004). Alzheimer's disease is the leading cause of dementia in the elderly. As the incidence and prevalence of Alzheimer's disease rise steadily with increasing longevity, Alzheimer's disease threatens to become a catastrophic burden on health care, particularly in developed countries [Alzheimer's Disease Education & Referral Center: http://www.nia.nih.gov/Alzheimers/AlzheimersInform-ation/GeneralInfo]. However, there are very few therapeutic drugs or interventions effective for treatment of Alzheimer's disease and/or other neurodegenerative disorders. As such, there is a need for developing a novel therapeutic strategy for treatment of neurodegenerative and/or neurological disorders, e.g., Alzheimer's disease and/or diseases or conditions where axonal growth and/or regeneration is desired to be promoted.

### SUMMARY

Embodiments of various aspects described herein stem, in part, from the discovery of osteocrin (OSTN), a secreted protein produced by cells of the osteoblast lineage, as a human-specific neural activity-regulated gene. It was discovered that osteocrin expression is regulated by neuronal activity, e.g., membrane depolarization, and/or calcium influx through neuronal voltage-sensitive calcium channels. Further, it was discovered that treatment of human neural cells with an osteocrin-inducing agent, e.g., a recombinant human OSTN protein, can increase neuronal connectivity, neuronal survival and/or axonal growth of the human neural cells, thus providing a therapeutic neuro-protective intervention for treatment of a neurodegenerative and/or neurological condition and/or disorder, e.g., Alzheimer's disease and/or diseases or conditions where axonal growth and/or regeneration is desired to be promoted.

Accordingly, one aspect provided herein relates to methods of increasing neuronal connectivity and/or neuronal survival of a population of neural cells. The method comprises contacting a population of neural cells (e.g., neural cells in need of increased neuronal connectivity) with a composition comprising an effective amount of an osteocrin-inducing agent. In some embodiments, the population of neural cells comprise human neural cells.

The osteocrin-inducing agent can be any agent that can directly or indirectly increase intracellular and/or extracellular (secreted) levels of osteocrin, e.g., human osteocrin. Examples of an osteocrin-inducing agent can include, but are not limited to, a recombinant osteocrin protein, peptide or a peptidomimetic thereof or a portion thereof (e.g., a peptide or a peptidomimetic comprising C-terminus of osteocrin), a recombinant osteocrin-encoding nucleic acid molecule, optionally operably linked to an expression vector (e.g., a viral vector and/or a plasmid vector), a small molecule that induces intracellular and/or extracellular expression of osteocrin, a small-molecule osteocrin analog or a prodrug thereof, a neural cell (e.g., a neural stem cell) engineered with enhanced intracellular and/or extracellular expression of osteocrin, and any combinations thereof.

In some embodiments, the osteocrin-inducing agent can comprise a recombinant osteocrin protein or a peptidomimetic thereof. In some embodiments, the recombinant osteocrin protein or peptidomimetic thereof can be a recombinant human osteocrin protein or a peptidomimetic thereof. In some embodiments, the recombinant human osteocrin protein or peptidomimetic thereof can refer to a protein encoded by an amino acid sequence of at least a portion of human osteocrin and displaying an ability to promote neuronal connectivity, neuronal survival and/or axonal growth of human neural cells. In some embodiments, the recombinant human osteocrin protein or peptidomimetic thereof can refer to a recombinant protein having at least 70% or more homology to human osteocrin and displaying an ability to promote neuronal connectivity, neuronal survival and/or axonal growth of human neural cells.

In some embodiments, the osteocrin-inducing agent can comprise a small molecule that can directly or indirectly induce intracellular and/or extracellular (secreted) expression of osteocrin, e.g., human osteocrin. In some embodiments, the small molecule that can directly or indirectly induce intracellular and/or extracellular expression of osteocrin, e.g., human osteocrin, are not known for treatment of a neurodegenerative and/or neurological disorder and/or condition, e.g., Alzheimer's disease. In one embodiment, the small molecule excludes a peroxisome proliferator-activated receptor gamma (PPAR-gamma) agonist, e.g., but not limited to, Troglitazone.

In some embodiments, the small molecule can comprise an osteocrin analog, e.g., an osteocrin mimimetics that can interact with a cell receptor to which an osteocrin protein generally binds, or a prodrug thereof. For example, the small molecule can comprise a ligand or an agonist for a natriuretic peptide clearance receptor.

In some embodiments, the small molecule can comprise a recombinant osteocrin-encoding nucleic acid molecule (e.g., DNA, RNA), optionally operably linked to an expression vector (e.g., a viral vector and/or a plasmid vector),

The inventors have discovered that induction of osteocrin expression by neuronal activity and/or depolarization occurs selectively in human neurons (not in, e.g., mouse neurons). In some embodiments, addition of brain-derived neurotrophic factor (BDNF), a neuronal activity-regulated secreted factor, in human cultures does not have any significant effect on induction of osteocrin expression. In some embodiments, OSTN expression can be induced only by depolarization.

Since osteocrin is a human-specific neural activity-regulated gene, where induction of osteocrin expression by neuronal activity and/or depolarization occurs selectively in human neurons (not in, e.g., mouse neurons), in some embodiments, an osteocrin-inducing agent for use in the methods and/or compositions described herein can comprise an agent or molecule that has been developed or discovered in non-human animal models (e.g., but not limited to, mouse or rat) for increasing osteocrin expression in non-neurons and/or non-brain tissues (e.g., but not limited to, bone or skeletal tissues). In some embodiments, an osteocrin-inducing agent for use in the methods and/or compositions described herein can comprise an osteocrin-inducing agent or molecule that has been developed in non-human animal models (e.g., but not limited to, mouse or rat) for treatment of non-neuronal diseases or disorders (e.g., but not limited to, bone diseases or disorders). Accordingly, in some embodiments, the methods and/or compositions described herein can represent a treatment that is tailored to features of neurons that are unique to human and therefore have not been previously targeted by osteocrin-inducing agents that have been developed using non-human animal models (e.g., but not limited to mouse or rat).

The method described herein can be performed in vitro, ex vivo, or in vivo. For example, in some embodiments, the population of neural cells (e.g., human neural cells) can be contacted with a composition comprising an osteocrin-inducing agent in vitro, e.g., in a cell culture. In some embodiments, the population of neural cells can be collected from a subject (e.g., a human subject) and cultured ex vivo before implantation into the subject.

In some embodiments, an osteocrin-inducing agent can be introduced to a population of neural cells in vivo. The population of neural cells can be present in a subject (e.g., a human subject) and thus be contacted with a composition comprising an osteocrin-inducing agent in vivo. In these embodiments, the subject amenable to the methods described herein can be a human subject. In some embodiments, a subject (e.g., a human subject) amenable to the methods described herein can be diagnosed as having, or having a risk for cognitive impairment, and/or a neurodegenerative condition and/or disorder. In some embodiments, the neurodegenerative condition and/or disorder can be present in the central nervous system. Exemplary neurodegenerative conditions and/or disorders can include, but are not limited to, Alzheimer's disease, Parkinson's disease, Huntington's disease, frontotemproal dementia, encephalitis, brain trauma, tau-associated neurodegenerative disorder, amyloid-beta-associated neurodegenerative disorder, inflammation-associated neurodegenerative disorder, and any disorder suffering from weakening synapses. In one embodiment, the subject amenable to the methods described herein can be a subject (e.g., a human subject) diagnosed with having, or having a risk for, Alzheimer's disease.

Neuronal and synaptic defects can be found in neuropsychiatric diseases or disorders. In accordance with various aspects described herein, OSTN can increase neuronal connectivity, survival and/or axonal growth. Accordingly, in some embodiments, a subject (e.g., a human subject) amenable to the methods described herein can be diagnosed as having, or having a risk for a neuropsychiatric disease and/or disorder. Examples of neuropsychiatric disease and/or disorder includes, without limitations, schizophrenia, depression, autism, autism spectrum disorders (ASDs), obsessive compulsive disorder, visual hallucination, auditory hallucination, eating disorder, bipolar disorder, and any combinations thereof. In some embodiments, the subject amenable to the methods described herein can be a subject (e.g., a human subject) diagnosed with having, or having a risk for, autism spectrum disorders (ASDs).

In some embodiments, the methods described herein can be used to treat a subject diagnosed as having, or having a risk for a motor neuron disease. Examples of motor neuron diseases are described herein.

Accordingly, methods of treating a subject (e.g., a human subject) diagnosed as having, or having a risk for, a neurodegenerative condition and/or disorder are also provided herein. For example, the method comprises administering to a subject (e.g., a human subject) a pharmaceutical composition comprising an effective amount of an osteocrin-inducing agent described herein. In some embodiments, the method can further comprise selecting a subject (e.g., a human subject) diagnosed as having, or having a risk for, cognitive impairment and/or a neurodegenerative condition prior to administration with a composition comprising an osteocrin-inducing agent.

In some embodiments, the methods of treatment described herein can be used to treat a disease or condition where axonal growth and/or regeneration is desired to be promoted in neural cells. Accordingly, in one aspect, methods of treating a subject in need of promoting axonal growth and/or regeneration of neural cells are provided herein.

In accordance with embodiments of various aspects described herein, any amounts of an osteocrin-inducing agent can be administered to a population of neural cells in vitro or in vivo, provided that the administered amounts do not induce any adverse effects on neural cells, e.g., decreased cell viability and/or function, e.g., neuronal connectivity and/or axonal growth. In some embodiments, a population of neural cells (e.g., in a cell culture or in a human subject) can be contacted with an effective amount of the osteocrin-inducing agent sufficient to increase the neuronal connectivity, neuronal survival and/or axonal growth of the population of neural cells (e.g., in a cell culture or in a human subject), e.g., by at least about 10% or more, as compared to a control population of neural cells not contacted or administered with the osteocrin-inducing agent.

The neuronal connectivity, neuronal survival and/or axonal growth of the population of neural cells can be measured and/or determined in vitro or in vivo by any methods known in the art. For example, in some embodiments of this aspect and other aspects described herein, the neuronal connectivity, neuronal survival and/or axonal growth of the population of neural cells can be determined and/or monitored with an imaging system, e.g., a microscope, and/or a functional magnetic resonance imaging, alone or in combination with use of at least one neural cell indicator.

In some embodiments, an increase in neuronal connectivity can be characterized and/or measured by various neuronal connectivity indicators. For example, in some embodiments, an increase in neuronal connectivity can be measured by determining an increase in dendritic density in the population of neural cells. In such embodiments, the effective amount of an osteocrin-inducing agent introduced to the neural cells can be an amount sufficient to increase the dendritic density in the population of neural cells by at least about 10% or more, as compared to a control population of neural cells not contacted with the osteocrin-inducing agent.

Additionally or alternatively, an increase in neuronal connectivity can be measured by determining an increase in excitatory synapse density in the population of neural cells. In such embodiments, the effective amount of an osteocrin-inducing agent can be an amount sufficient to increase the excitatory synapse density in the population of neural cells by at least about 10% or more, as compared to a control population of neural cells not contacted with the osteocrin-inducing agent.

Methods to detect changes in dendritic density and/or excitatory synapse density in a population of neural cells are known in the art and can be utilized in the methods described herein. In some embodiments of this aspect and other aspects described herein, increases in the dendritic and excitatory synapse densities can be measured by determining, e.g., with immunostaining, an increased expression of at least one or more dendritic and synaptic (e.g., post-synaptic) markers, respectively. Non-limiting examples of dendritic marker and/or synaptic marker include MAP2, PSD95, synapsin, and any combinations thereof.

In some embodiments of this aspect and other aspects described herein, an increase in neuronal connectivity can be measured by determining an increase in neuronal survival in the population of neural cells. In such embodiments, the effective amount of an osteocrin-inducing agent can be an amount sufficient to increase the neuronal survival in the population of neural cells by at least about 10% or more, as compared to a control population of neural cells not contacted with the osteocrin-inducing agent.

Methods to detect changes in neuronal survival of neural cells are known in the art and can be used in the methods described herein. In some embodiments of this aspect and other aspects described herein, an increase in neuronal survival of neural cells can be measured by determining, e.g., with immunostaining, an increased number of cells that are positive for at least one dendritic marker, e.g., but not limited to MAP2.

In some embodiments of this aspect and other aspects described herein, an increase in neuronal connectivity can be measured by determining an increase in axonal growth of neural cells. In these embodiments, the effective amount of an osteocrin-inducing agent can be an amount sufficient to increase the length of at least one axon of neural cells by at least about 10% or more, as compared to a control population of neural cells not contacted with the osteocrin-inducing agent. The axonal growth of neural cells can be detected by any methods known in the art. In some embodiments, the axons of neural cells can be identified by the presence of at least one or more axonal markers. An exemplary axonal marker is a marker for neurofilament. In some embodiments, the axons of neural cells do not exhibit a dendritic marker.

In some embodiments where the methods of various aspects described herein are performed in vivo, e.g., in a subject, an increase in neuronal connectivity can be measured by determining an increase in cognitive function of the subject. In such embodiments, the effective amount of the osteocrin-inducing agent can be an amount sufficient to increase the cognitive function of the subject by at least about 10% or more, as compared to a control subject not administered with the osteocrin-inducing agent.

In some embodiments, the osteocrin-inducing agent can be introduced or delivered to a population of neural cells (e.g., human neural cells) in vitro or in vivo at an effective amount of about 0.1 ng/mL to about 100 μg/mL, or from about 0.5 ng/mL to about 50 ng/mL.

In some embodiments, the osteocrin-inducing agent can be administered to a subject at an effective amount of about 0.1 ng/kg to about 500 mg/kg body mass of the subject. In some embodiments, the osteocrin-inducing agent can be administered to a subject at an effective amount of about 1 mg/kg to about 250 mg/kg body mass of the subject. In some embodiments, the osteocrin-inducing agent can be administered to a subject at an effective amount of about 50 mg/kg to about 150 mg/kg body mass of the subject.

In some embodiments of this aspect and other aspects described herein, the composition or pharmaceutical composition can further comprise one or more neural stem cells. In some embodiments, the neural stem cells can be engineered to express and/or secrete osteocrin or an analog thereof at a level higher than (e.g., at least 5% higher than) that of control neural cells, e.g., target neural cells to be treated.

In some embodiments of this aspect and other aspects described herein, the composition or pharmaceutical composition can further comprise at least one or more therapeutic agents for treatment of a neurodegenerative and/or neurological condition and/or disorder described herein. In some embodiments, the composition or pharmaceutical composition can further comprise at least one or more therapeutic agents known to promote neuronal survival, axonal growth and/or regeneration of neural cells.

Without wishing to be bound by theory, as osteocrin is a neural activity-regulated gene, particularly in a human subject, osteocrin can be used as a neural biomarker to determine if a subject has, or has a risk for developing a neurodegenerative condition and/or disorder. Accordingly, a further aspect described herein relates to an assay for determining a subject's susceptibility to, or risk for, developing a neurodegenerative condition and/or disorder. The assay comprises (a) subjecting a test sample of a subject, who is determined to have, or have displayed symptoms of cognitive impairment, to at least one analysis to determine expression of osteocrin; (b) comparing the expression of osteocrin with a reference value using a non-human machine; and (c) optionally administering to the subject an osteocrin-inducing agent if the comparison indicates that a subject is diagnosed as having, or having a risk for, a neurodegenerative condition and/or disorder.

In some embodiments, the reference value can correspond to expression of osteocrin in a normal healthy subject. In such embodiments, a lower expression of osteocrin determined in the test sample of the subject than that of the normal healthy subject, e.g., by at least about 10% or more, can be indicative of the subject diagnosed as having, or having a risk for a neurodegenerative condition and/or disorder.

In other embodiments, the reference value can correspond to expression of osteocrin in a control subject diagnosed with a neurodegenerative condition and/or disorder. In such embodiments, a higher expression of osteocrin determined in the test sample of the subject than that of the control subject, e.g., by no more than 30%, can be indicative of the subject diagnosed as having, or having a risk for, a neurodegenerative condition and/or disorder.

In some embodiments, the reference value can correspond to osteocrin expression of a test sample collected from the same subject at a prior time point. In such embodiments, a decreased level of osteocrin expression in a test sample collected from the same subject collected at a later time point, relative to the prior time point measurement, can be indicative of the subject diagnosed as having, or having a risk for, a neurodegenerative condition and/or disorder.

In some embodiments, the test sample to be analyzed can comprise a blood sample collected from a subject (e.g., a human subject). In some embodiments, the test sample to be analyzed can comprise a brain biopsy obtained from a subject (e.g., a human subject).

Expression of osteocrin in a test sample of a subject can be determined by any analyses known in the art, depending on, e.g., types of test samples and/or abundance of osteocrin in test samples. For example, without limitations, western blot, enzyme linked absorbance assay, mass spectrometry, immunoassay, flow cytometry, immunohistochemical analysis, PCR reaction, real-time quantitative PCR, and any combinations thereof, can be used to determine expression (e.g., protein-level and/or transcript-level) of osteocrin in test samples.

The assay described herein can be used to diagnose a subject (e.g., a human subject) suspected to have, or have a risk for having, neurodegenerative conditions and/or disorders. Additionally or alternatively, the assay described herein can be used to monitor the progression of a neurodegenerative conditions and/or disorders, and/or efficacy of the corresponding treatment. Examples of neurodegenerative conditions and/or disorders include, but are not limited to, Alzheimer's disease, Parkinson's disease, Huntington's disease, frontotemproal dementia, encephalitis, brain trauma, tau-associated neurodegenerative disorder, amyloid-beta-associated neurodegenerative disorder, inflammation-associated neurodegenerative disorder, and any disorder suffering from weakening synapses. In one embodiment, the assay described herein can be used to diagnose Alzheimer's disease. In one embodiment, the assay described herein can be used to diagnose a condition in which axonal growth and/or regeneration is desired to be promoted in neural cells.

Cell-culture, pharmaceutical and/or neuro-protective compositions comprising an effective amount of an osteocrin-inducing agent for use in increasing neuronal connectivity, neuronal survival and/or axonal growth of a population of neural cells are also provided herein. These compositions can be employed in the methods and/or assays of various aspects described herein, e.g., for in vitro applications or in vivo applications such as treatment, diagnosis and/or monitoring of a neurodegenerative condition and/or disorder in a subject, e.g., a human subject.

In another aspect, methods for increasing neuronal survival of a population of neural cells are provided herein. The methods comprise contacting the population of neural cells with a composition comprising an effective amount of an osteocrin-inducing agent described herein.

In yet another aspect, methods for increasing axonal growth of a population of neural cells are provided herein. The methods comprise contacting the population of neural cells with a composition comprising an effective amount of an osteocrin-inducing agent described herein.

### BRIEF DESCRIPTION OF THE DRAWINGS

FIG. 1 is a fluorescent image showing an in vitro culture system for primary human cortical cells. Primary fetal human cortical cultures (GSW20) were maintained for about 6 days in vitro, and then fixed, and immunostained with a neuronal marker, e.g., MAP2.

FIGS. 2A-2B are bar graphs showing changes in osteocrin expression levels in human and mouse in response to activity of neural cells, indicating that osteocrin is a human-specific neural activity-regulated gene. FIG. 2A is a bar graph showing that membrane depolarization induces a dramatic increase in OSTN mRNA in human neurons but not in mouse neurons as quantified through RNA-sequencing. FIG. 2B is a bar graph showing data of RT-PCR experiments on induction of OSTN expression (normalized to GAPDH expression) in response to activity in human neurons. The data are represented as mean±SEM from n=3.

FIGS. 3A-3E are experimental data showing that osteocrin promotes human neuronal connectivity. FIGS. 3A-3D are fluorescent images of primary human neurons fixed and immunostained with neuronal and synaptic markers, e.g., microtubule-associated protein 2 (MAP2), postsynaptic density protein-95 (PSD95) and synapsin after cultured for about 10 days in vitro with and without recombinant OSTN (˜50 ng/mL). As compared to the control neurons (not contacted with recombinant OSTN), the human neurons treated with recombinant OSTN formed a denser network of microtubules, and expressed higher levels of PSD95 and synapsins. FIG. 3E is a bar graph showing quantification results of excitatory synapse number in human neuronal cultures with increasing concentrations (˜0.5-˜50 ng/mL) of recombinant OSTN. In particular, the human neuronal cultures were treated with recombinant OSTN at a concentration of ˜0.5 ng/mL, ˜5 ng/mL, or ˜50 ng/mL. The data are represented as mean±SEM from n=5.

FIGS. 4A-4C are experimental data showing that osteocrin promotes human neuronal survival. FIGS. 4A-4B are fluorescent images showing culture of primary human neurons with or without recombinant OSTN. In FIGS. 4A-4B, primary human neurons were cultured for 10 days in vitro with and without recombinant OSTN (˜50 ng/ml), fixed and immunostained with neuronal marker MAP2 and nuclear marker DAPI. FIG. 4C is a quantitative bar graph showing the number of MAP2 positive neurons per measured area at DIV10 in human neuronal cultures with or without recombinant OSTN. The data are represented as mean±SEM. As used herein throughout the specification, the abbreviation “DIV” stands for Day In Vitro, i.e., the number of days neurons have grown in culture.

FIGS. 5A-5G are experimental data showing that osteocrin promotes human axonal growth. FIG. 5A is a schematic diagram of microfluidic chambers, e.g., for use in a human axonal growth assay. The bracket denotes the area where images were approximately acquired. In FIGS. 5B-5E, primary human neurons were cultured for 21 days in vitro in the microfluidic chamber with (FIG. 5C and FIG. 5E) and without (FIG. 5B and FIG. 5D) recombinant OSTN (˜100 ng/ml). FIGS. 5B-5C are brightfield images of axons imaged live by a bright-field microscope. FIGS. 5D-5E are fluorescent images of axons fixed and immunostained with axonal marker Neurofilament (NF). FIG. 5F is a quantitative bar graph showing axonal length extension of OSTN-treated neurons, normalized to the control experiment. FIG. 5G is a quantitative bar graph showing the number of MAP2-positive neurons per measured area inside the chamber. The data are represented as mean±SEM.

### DETAILED DESCRIPTION OF THE INVENTION

Neurodegenerative and/or neurological disorders, e.g., Alzheimer's disease, affect millions of individuals. While loss of neuronal connectivity in a central nervous system has been correlated with cognitive impairments in various neurodegenerative conditions, e.g., Alzheimer's disease, there are yet no or very few therapeutic drugs or interventions effective for increasing neuronal connectivity in the neural cells that can be used for treatment of these devastating disorders, e.g., Alzheimer's disease. Accordingly, there is a need for developing or identifying a novel therapeutic agent or strategy for treatment of these neurodegenerative and/or neurological disorders, e.g., Alzheimer's disease.

To this end, the inventors have surprisingly discovered inter alia that osteocrin (OSTN), which is traditionally known as a secreted protein produced by cells of osteoblast lineage and associated primarily with modulation of bone growth, can unexpectedly increase neuronal connectivity of neural cells (e.g., human neural cells), e.g., as evidenced by a significant increase in excitatory synapse density, dendritic density and/or total dendritic length, as compared to the control neural cells without treatment of the osteocrin-inducing agent. Additionally, the inventors have discovered that OSTN can unexpectedly increase neuronal survival and/or axonal growth of neural cells (e.g., human neural cells). Further, osteocrin is discovered to be a human-specific neural activity-regulated gene. For example, OSTN expression can be regulated by neural activity of human neural cells such as membrane depolarization, and/or calcium influx through neuronal voltage-sensitive calcium channels). Thus, delivery of OSTN to neural cells, e.g., human neural cells, can promote their neuronal connectivity, neuronal survival and/or axonal growth, which can be utilized to provide a therapeutic neuro-protective intervention for treatment of various neurodegenerative and/or neurological conditions, e.g., but not limited to, Alzheimer's disease, and/or for treatment of diseases or conditions in which axonal growth and/or regeneration is desired to be promoted. Accordingly, embodiments of various aspects described herein generally relate to methods and compositions for increasing neuronal connectivity, neuronal survival and/or axonal growth in vitro or in vivo. Methods and compositions for treatment and/or diagnosis of cognitive impairment or a neurodegenerative condition (e.g., Alzheimer's disease) are also provided herein. In one aspect, methods and compositions for increasing neuronal survival in vitro or in vivo are also provided. In another aspect, methods and compositions for increasing axonal growth of neural cells in vitro or in vivo are also provided.

Methods of Increasing Neuronal Connectivity, Neuronal Survival and/or Axonal Growth of Neurons; and Treatment of Neurodegeneration

In one aspect, provided herein are methods of increasing neuronal connectivity of a population of neural cells. The method comprises contacting a population of neural cells (e.g., neural cells in need of increased levels of neuronal connectivity and/or neuronal survival) with a composition comprising an effective amount of an osteocrin-inducing agent. In some embodiments, the population of neural cells can comprise human neural cells.

In another aspect, methods of increasing neuronal survival of a population of neural cells are also provided herein. The method comprises contacting a population of neural cells (e.g., neural cells in need of increased levels of neuronal connectivity and/or neuronal survival) with a composition comprising an effective amount of an osteocrin-inducing agent.

Methods of regenerating axons or increasing axonal growth of a population of neural cells are also provided herein. The method comprises contacting a population of neural cells (e.g., neural cells in need of axonal growth and/or regeneration) with a composition comprising an effective amount of an osteocrin-inducing agent.

As used herein, the term “neural cells,” also known by the art-recognized term “neurons,” refers to cells that express one or more neuron-specific markers. Examples of such markers can include, but are not limited to, neurofilament, microtubule-associated protein-2 (MAP2), tau protein, neuron-specific Class III β-tubulin, and NeuN. In some embodiments, neural cells can include cells that are post-mitotic and express one or more neuron-specific markers. In some embodiments, the neural cells can be present or obtained from the nervous system, which includes, e.g., the brain, spinal cord, and/or peripheral ganglia. Neural cells can include various specialized types of neurons including, e.g., but not limited to, sensory neurons (responsive to touch, sound, light and/or numerous other stimuli and affecting cells of the sensory organs that then send signals to the spinal cord and brain), motor neurons (capable of receiving signals from the brain and spinal cord, causing muscle contractions, and affecting glands), interneurons (connecting neurons to other neurons within the same region of the brain or spinal cord), or any combinations thereof. In some embodiments, neural cells can include neural stem cells. In some embodiments, the term “neural cells” refer to human neural cells.

The term “population” as used herein refers to at least two or more cells sharing at least one or more identical phenotypic characteristics. In some embodiments, the term “population” refers to at least two or more cells having substantially identical phenotypic characteristics. The phrase “a population of neural cells” as used herein refers to a collection of cells, in which at least two or more cells express at least one neuron-specific markers, for example, at least about 10% neural cells, at least about 20% neural cells, at least about 30% neural cells, at least about 40% neural cells, at least about 50% neural cells, at least about 60% neural cells, at least about 70% neural cells, at least about 80% neural cells, at least about 90% neural cells, at least about 95% neural cells, at least about 98% neural cells, at least about 99% neural cells, or 100% neural cells. Other cells that can be present in a population of neural cells can include, but are not limited to, cells that can provide and/or protect neural cells, e.g., non-neural cells such as glia cells. Glia cells can include, e.g., but are not limited to, oligodendrocytes, microglia, and/or astrocytes, the presence of which can optimize brain function.

As used herein, the term “contacting” refers to any suitable means for delivering, or exposing, an osteocrin-inducing agent to a population of neural cells. Exemplary delivery methods include, but are not limited to, direct delivery (e.g., direct addition) of an osteocrin-inducing agent to cell culture medium in which the neural cells are cultured in vitro or ex vivo; delivery to an in vitro scaffold in which cells are seeded, e.g., via perfusion and/or injection; delivery to neural cells in vivo, e.g., in a tissue of a subject, e.g., by injection and/or implantation (e.g., direct placement); and/or other delivery method well known to one skilled in the art.

In some embodiments, the neural cells can be contacted with an osteocrin-inducing agent in vitro. For example, where the neural cells are present in a cell culture, the neural cells can be contacted with an osteocrin-inducing agent by adding the osteocrin-inducing agent to the cell culture medium in which neural cells are cultured. Alternatively, an osteocrin-inducing agent can be injected into a biocompatible gel (e.g., peptide gel, hydrogel) in which neural cells are embedded and cultured. In some embodiments, the cell culture can comprise a population of human neural cells.

In some embodiments, the neural cells can be contacted with an osteocrin-inducing agent ex vivo. The term “ex vivo” refers to a condition where biological materials, e.g., neural cells, obtained from a subject or a suitable alternate source, such as, suitable donor, are cultured outside the subject in an environment similar to a physiological condition (e.g., an environment with minimum alteration of the physiological condition). For example, neural cells and/or neural stem cells obtained from a subject (e.g., a human subject) or an alternative source can be treated with an osteocrin-inducing agent (e.g., for increasing neuronal connectivity, neuronal survival and/or axonal growth), and then re-introduced into the subject. In these embodiments, an ex vivo therapy can provide a subject (e.g., a human subject) or a patient the benefit of the treatment, without exposing the patient to undesired collateral effects, if any, from the treatment. The term “treatment” or “treated” in reference to exposing cells to an agent, e.g., treatment of neural cells with an osteocrin-inducing agent, is used herein interchangeably with the term “contacting.”

In some embodiments, the neural cells can be contacted with an osteocrin-inducing agent in vivo, e.g., in a subject. In these embodiments, an osteocrin-inducing agent can be administered to a human subject in need of an osteocrin-inducing agent. In some embodiments, the human subject in need of an osteocrin-inducing agent can be a human subject diagnosed with having, or having a risk for, cognitive impairment and/or a neurodegenerative condition, e.g., but not limited to, Alzheimer's disease. In other embodiments, the human subject in need of an osteocrin-inducing agent can be a human subject in need of axonal growth and/or regeneration in his/her neural cells.

An osteocrin-inducing agent can be administered to a subject by any art-recognized methods. In some embodiments, an osteocrin-inducing agent can be administered to a subject by injection, e.g., but not limited to, intracortical injection. Other forms of administration can also be employed in the methods described herein, e.g., systemic, inhalation, oral, parenteral, and/or implantation or direct placement (e.g., by surgery). One of skill in the art can determine an appropriate administration method known in the art based on, e.g., location of target sites to be treated, and/or formulations of an osteocrin-inducing agent (e.g., but not limited to, a spray, a solution, a tablet, and any combination thereof).

For example, in some embodiments where the neural cells are present in the brain of a subject, an osteocrin-inducing agent can be delivered to the neural cells or administered to the subject by injection. In one embodiment, the injection is intracortical. Alternatively, the neural cells can be contacted with an osteocrin-inducing agent by intracranial injection. In some embodiments, the injection can be performed by a catheter-based approach, e.g., with or without imaging. The use of a catheter can preclude more invasive methods of delivery where the opening of the brain would be necessitated. As one skilled in the art would appreciate, optimum time of recovery would be allowed by the more minimally invasive procedure.

In some embodiments, at least one or more osteocrin-inducing agent can be administered to a subject intravenously.

In some embodiments, at least one or more osterocrin-inducing agent can be intrathecally administered. For example, where one targets motor neural cells in a subject, intrathecal administration of at least one osteocrin-inducing agent can be performed.

In some embodiments, the population of neural cells described herein can be contacted more than once with at least one osteocrin-inducing agent over a pre-determined period of time, e.g., in hours, days, weeks, months or years. In some embodiments, the neural cells can be contacted with one or more osteocrin-inducing agents at least twice, at least three times, at least four times, or at least five times over a pre-determined period of time, e.g., in hours, days, weeks, months, or years. The same or a different osteocrin-inducing agent can be used in each treatment of neural cells. The treatment or administration frequency can vary depending on, e.g., half-life, potency, toxicity window, and/or concentration of the osteocrin-inducing agent administered, degree of neurodegeneration of neural cells (in vitro or in vivo), the population size of neural cells, physical and/or clinical conditions of a subject (if in vivo), and any combinations thereof.

In some embodiments, the neural cells can be further contacted with at least one or more active agents prior to, concurrent with, or after the treatment with at least one osteocrin-inducing agent. Such active agents can include, but are not limited to, proteins, peptides, antigens, antibodies or portions thereof, antibody-like molecules, enzymes, nucleic acids, siRNA, shRNA, aptamers, small molecules, antibiotics, therapeutic agents (e.g., for treatment of neurodegeneration or a neurodegenerative condition, e.g., Alzheimer's disease, and/or for promotion of axonal growth and/or regeneration), contrast agents, cytokines, neural growth factors, anesthetics, an additional osteocrin-inducing agent, or a mixture thereof. In one embodiment, the neural cells can be contacted with at least one or more (e.g., at least two, at least three or more) therapeutic agents prior to, concurrent with, or after the treatment with at least one osteocrin-inducing agent. The therapeutic agents can include therapeutic agents for treatment of neurodegeneration or a neurodegenerative condition, e.g., but not limited to, Alzheimer's disease, and/or for promotion of axonal growth and/or regeneration.

Methods of Treatment:

In some embodiments, the methods described herein can be performed in vivo to increase neuronal connectivity, the loss of which in the central nervous system has been correlated with cognitive impairments in various neurodegenerative conditions, e.g., Alzheimer's disease. In some embodiments, the methods described herein can be performed in vivo to increase neuronal survival in a subject, e.g., a human subject. In some embodiments, the methods described herein can be performed in vivo to increase axonal growth and/or regeneration in a subject, e.g., a human subject. Without wishing to be bound by theory, some embodiments of the methods described herein can be used to treat and/or prevent cognitive impairment and/or a neurodegenerative condition e.g., but not limited to, Alzheimer's disease, in a subject (e.g., a human subject). Accordingly, another aspect provided herein relates to methods of treating a subject (e.g., a human subject) diagnosed with having, or having a risk for, cognitive impairment, and/or a neurodegenerative condition and/or disorder. For example, the method comprises administering to a subject (e.g., a human subject) a pharmaceutical composition comprising an effective amount of an osteocrin-inducing agent. In some embodiments, the method of treatment can further comprise selecting a subject (e.g., a human subject) who is determined to have, or have a risk for, cognitive impairment and/or a neurodegenerative condition, e.g., but not limited to, Alzheimer's disease, prior to administration with a pharmaceutical composition comprising an osteocrin-inducing agent.

As used herein, the term “cognitive impairment” refers to a deterioration or loss of mental function, e.g., abnormalities of thinking and memory, that are associated with temporary or permanent brain dysfunction, e.g., caused by or associated with an acquired brain lesion and/or a neurodegenerative disease or disorder. Their main symptoms include, but are not limited to, problems with memory, orientation, language, information processing, and the ability to focus and sustain attention on a task. Cognitive impairment is typically manifested by one or more cognitive deficits. Memory impairment is a cognitive deficit characterized by the inability to learn new information or to recall previously learned information. Aphasia is a cognitive deficit characterized by a language and/or speech disturbance. Apraxia is a cognitive deficit characterized by the impaired ability to carry out motor activities despite intact motor function according to DSM-TV. Agnosia is a cognitive deficit characterized by the failure to recognize or identify objects despite intact sensory function (as described in DSM-TV). Cognitive impairment may also be manifested by a disturbance in executive functioning (i.e., planning, organizing, sequencing, abstracting).

Cognitive impairment can be generally categorized as vascular dementia, neurodegenerative cognitive impairment, and exogenous cognitive impairment based on the causes thereof. These diseases can occur simultaneously, which is referred to as mixed dementia. For example, the mixed dementia can be caused by the Alzheimer's dementia and vascular dementia. Cognitive impairment can also include mild cognitive impairment (MCI), i.e. a prior stage in which the symptom may develop to various types of dementia. Vascular dementia means dementia caused by cerebrovascular disorder, cerebral infarction or intracerebral hemorrhage. Representative examples of neurodegenerative cognitive impairment can include, e.g., but not limited to, Alzheimer's dementia and non-Alzheimer's dementia. It is known that non-Alzheimer's dementia includes, for example, dementia with Lewy body, neurofibrillary tangle dementia, Parkinson disease, Huntington disease, frontotemporal dementia [Pick disease, progressive subcortical gliosis (PSG), amyotrophic lateral sclerosis with dementia (ALS-D), frontal lobe dementia, frontotemporal dementia and parkinsonism linked to chromosome 17 (FTDP-17)], dementia with glial tangles [progressive supranuclear palsy (PSP), corticobasal degeneration (CBD)], argyrophilic grain disease, dementias with predominant degeneration in subcortical nucleus [Huntington disease, dentatorubral-pallidoluysian atrophy (DRPLA), thalamic degeneration], and other types of degenerative dementia difficult to be classified. Exogenous cognitive impairment includes diseases caused by, for example, schizophrenia, depression, bipolar depression, diabetes, attention deficit hyperactivity disorder, Creutzfeldt-Jakob disease, Kraepelin disease, Hallervorden-Spatz disease, spinocerebellar ataxia, progressive myoclonus epilepsy, progressive supranuclear palsy, viscous edema, parathyroid disease, Wilson disease, hepatic disease, hypoglycemia, remote symptoms of cancer, Cushing syndrome, uremia, arteriosclerosis, cerebral arteriosclerosis, chronic cerebral circulatory insufficiency, intracerebral hemorrhage, cerebral infarction, cerebral embolism, subarachnoid hemorrhage, chronic subdural hemorrhage, pseudobulbar palsy, aortic arch syndrome, Binswanger disease, arteriovenous malformation—thromboangiitis obliterans, hypoxia, anoxia, normal pressure hydrocephalus, Wernicke-Korsakoff syndrome, pellagra, Marchiafava-Bignami disease, vitamin B 12 deficiency, disorders caused by metals, organic compounds, carbon monoxide, toxicants or drugs, brain tumor, open and closed head injury, Banti syndrome, fever attack, infection, bacterial meningitis, fungal meningitis, encephalitis, progressive multifocal leukoencephalopathy, Behcet syndrome, Kuru disease, syphilis, multiple sclerosis, muscular dystrophy, Whipple disease, concentration camp syndrome, disseminated lupus erythematosus, cardiac arrest, AIDS encephalopathy, hypothyroidism, hypopituitarism, and chronic alcoholism.

As used herein, the term “neurodegenerative condition” refers to any condition, disease or disorder caused by or associated with deterioration of neural cells or neurons, for example, where neuronal structure and/or function of neural cells or neurons is reduced, including death of neurons. The term “neurodegenerative condition” encompasses acute and chronic conditions, disorders or diseases of the central or peripheral nervous system. A neurodegenerative condition can be age-related, or it can result from injury or trauma, or it can be related to a specific disease or disorder. Acute neurodegenerative conditions include, but are not limited to, conditions associated with neuronal cell death or compromise including cerebrovascular insufficiency, e.g., due to stroke, focal or diffuse brain trauma, diffuse brain damage, spinal cord injury or peripheral nerve trauma, e.g., resulting from physical or chemical burns, deep cuts or limb severance. Examples of acute neurodegenerative disorders include, without limitations, cerebral ischemia or infarction including embolic occlusion and thrombotic occlusion, reperfusion following acute ischemia, perinatal hypoxic-ischemic injury, cardiac arrest, as well as intracranial hemorrhage of any type (such as epidural, subdural, subarachnoid and intracerebral), and intracranial and intravertebral lesions (such as contusion, penetration, shear, compression and laceration), as well as whiplash and shaken infant syndrome. Chronic neurodegenerative conditions include, but are not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis (ALS), degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal muscular atrophy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (including, but not limited to Creutzfeldt-Jakob, Gerstmann-Straussler-Scheinker disease, Kuru and fatal familial insomnia), demyelination diseases and disorders including multiple sclerosis, spinal cord-related injuries, hereditary diseases such as leukodystrophies, and any disorder suffering from weakening synapses. In one embodiment, the subject amenable to the methods described herein can be a subject (e.g., a human subject) diagnosed with having, or having a risk for, Alzheimer's disease.

In some embodiments, the neurodegenerative conditions to be treated with the methods described herein can include neuronopathies. The term “neuronopathies” as used herein refers to diseases or disorders that are characterized by neuronal cell death of motor neurons or sensory neurons and hence neuronopathies can be subdivided in motor and sensory neuron disorders. Motor Neuron Disease (MND) or motor neuron disorders is a group of diseases (disorders) involving the degeneration of the anterior horn cells, nerves in the central nervous system that control muscle activity. This leads to gradual weakening and eventually wasting of the musculature (atrophy). Diseases of the motor neuron are generally classified according to upper motor neuron (UMN) and/or lower motor neuron (LMN) involvement. Upper motor neurons originate in the brain, in particular, the motor cortex, and they synapse either directly or indirectly onto lower motor neurons. Upper motor neurons are more accurately referred to as pre-motor neurons, and they are responsible for conveying descending commands for movement. Lower motor neurons are divided into two categories: visceral and somatic motor neurons. Visceral motor neurons are autonomic pre-ganglionic neurons that regulate the activity of ganglionic neurons, which innervate glands, blood vessels, and smooth muscle. Somatic motor neurons innervate skeletal muscle and include first, anterior horn cells, which as the name implies, are located in the anterior horn of the spinal cord, and second, lower motor neurons located in the cranial nerve nuclei. Amyotrophic lateral sclerosis or ALS, also known as Lou Gehrig's disease, is the most frequent form (accounting for around 80% of all cases) of motor neuron disorders. The initial symptoms of ALS are weakness in the hands and legs and often fasciculation of the affected muscles. Whichever limbs are affected first, all four limbs are affected eventually. Damage to the upper motor neurons produces muscle weakness, spasticity and hyperactive deep tendon reflexes. Lower motor neuron damage produces muscle weakness with atrophy, fasciculation, flaccidity and decreased deep tendon reflexes. ALS has features of both upper and lower motor neurons of the cranial nerves; therefore symptoms are isolated to the head and neck. Some patients will also display UMN involvement of the cranial nerves and if this is the sole manifestation it is referred to as Pseudobulbar palsy. Spinal muscular atrophy or progressive muscular atrophy is a MND that does not involve the cranial nerves and is due to lower motor neuron degeneration. Shy-Drager syndrome is characterized by postural hypotension, incontinence, sweating, muscle rigidity and tremor, and by the loss of neurons from the thoracic nuclei in the spinal cord from which sympathetic fibres originate. Destructive lesions of the spinal cord result in the loss of anterior horn cells. This is seen in myelomeningocele and in syringomyelia, in which a large fluid-filled cyst forms in the center of the cervical spinal cord. Poliomyelitis virus infection also destroys anterior horn cells. Spinal cord tumors may locally damage anterior horn cells either by growth within the cord (gliomas) or by compression of the spinal cord from the outside (meningiomas, schwannomas, metastatic carcinoma, lymphomas).

In some embodiments, neuronopathies can include diseases or disorders where dorsal root ganglion cells are damaged, for example, by herpes simplex and varicella-zoster viruses. Such infections are associated with a vesicular rash in the skin regions supplied by those neurons. A similar loss of sensory neurons can be observed in ataxia telangiectasia, a disorder associated with progressive cerebellar ataxia and symmetrical telangiectasia of the skin and conjunctiva. Neuronal loss from autonomic ganglia can be observed in amyloid neuropathies and in diabetes.

In some embodiments, the neurodegenerative conditions to be treated with the methods described herein can include neuropsychiatric diseases and/or disorders. The term “neuropsychiatric disease and/or disorder” generally refers to a neuron-related (e.g., in brain) disease or dysfunction that causes psychiatric symptoms. Such diseases or disorders can also include “major mental illness disorder” or “major mental illness,” which refers to a disorder generally characterized by one or more breakdowns in the adaptation process. Such disorders are therefore expressed primarily in abnormalities of thought, feeling and/or behavior producing either distress or impairment of function (i.e., impairment of mental function such with dementia or senility). Methods to evaluate individuals for various neuropsychiatric disorders are known in the art, e.g., using criteria set forth in the most recent version of the American Psychiatric Association's Diagnostic and Statistical Manual of Mental Health (DSM-IV). Examples of neuropsychiatric disease and/or disorder includes, without limitations, schizophrenia, attention deficit disorder (ADD), schizoaffective disorder, depression, autism, autism spectrum disorders (ASDs), obsessive compulsive disorder, visual hallucination, auditory hallucination, eating disorder, bipolar disorder, and any combinations thereof. In some embodiments, the methods described herein can be used to treat a subject (e.g., a human subject) diagnosed with having, or having a risk for, autism spectrum disorders (ASDs).

The terms “treatment” and “treating” as used herein, with respect to treatment of a disease, means preventing the progression of the disease, or altering the course of the disorder (for example, but are not limited to, slowing the progression of the disorder), or reversing a symptom of the disorder (e.g., slowing and/or reversing neuronal atrophy) or reducing one or more symptoms and/or one or more biochemical markers in a subject, preventing one or more symptoms from worsening or progressing, promoting recovery or improving prognosis. For example, in the case of treating a neurodegenerative disorder, e.g., but not limited to, Alzheimer's disease, therapeutic treatment refers to reduced neurodegenerative morphologies, e.g., reduced neurite dystrophies, and/or improved cognitive function, after administration of the composition described herein. In another embodiment, the therapeutic treatment refers to alleviation of at least one symptom associated with a neurodegenerative disorder, e.g., but not limited to, Alzheimer's disease. Measurable lessening includes any statistically significant decline in a measurable marker or symptom, such as assessing the cognitive improvement with neuropsychological tests such as verbal and perception after treatment. In one embodiment, at least one symptom of a neurodegenerative disorder, e.g., but not limited to, Alzheimer's disease, is alleviated by about 10%, about 15%, about 20%, about 30%, about 40%, or about 50%. In another embodiment, at least one symptom is alleviated by more than 50%, e.g., about 60%, or about 70%. In one embodiment, at least one symptom is alleviated by about 80%, or about 90%, as compared to a control (e.g. in the absence of the composition described herein).

Suitable regimes for initial administration and further doses or for sequential administrations can be varied. In one embodiment, a therapeutic regimen includes an initial administration followed by subsequent administrations, if necessary. In some embodiments, multiple administrations of an osteocrin-inducing agent can be delivered to degenerating neural cells, e.g., by injection into the subject's brain. For example, an osteocrin-inducing agent can be administered in two or more, three or more, four or more, five or more, or six or more injections. In some embodiments, the same osteocrin-inducing agent can be administered in each subsequent administration. In some embodiments, a different osteocrin-inducing agent described herein can be administered in each subsequent administration. Injections can be made in cortex, e.g., somatosensory cortex.

A subsequent administration can be performed immediately after the previous administration, or after at least about 1 minute, after at least about 2 minute, after at least after about 5 minutes, after at least about 15 minutes, after at least about 30 minutes, after at least about 1 hour, after at least about 2 hours, after at least about 3 hours, after at least about 6 hours, after at least about 12 hours, after at least about 24 hours, after at least about 2 days, after at least about 3 days, after at least about 4 days, after at least about 5 days, after at least about 6 days or after at least about 7 days or longer. In some embodiments, the subsequent administration can be performed after at least about 1 week, at least about 2 weeks, at least about 3 weeks, at least about 1 month or longer. In some embodiments, the subsequent administration can be performed after at least about 1 year, at least about 2 years, at least about 3 years, at least about 5 years, at least about 10 years or longer.

In accordance with some embodiments described herein, a subject in need thereof, e.g., a human subject diagnosed with having, or having a risk for, cognitive impairment and/or a neurodegenerative condition; or a human subject in need of axonal growth and/or regeneration of his/her neural cells, is administered with a pharmaceutical composition comprising an effective amount of an osteocrin-inducing agent. In some embodiments, a dosage comprising an osteocrin-inducing agent is considered to be pharmaceutically effective if the dosage reduces degree of neurodegeneration, e.g., indicated by changes in neurodegenerative morphologies or improvement in brain or cognitive function, by at least about 5%, at least about 10%, at least about 15%, at least about 20%, at least about 30%, at least about 40%, or at least about 50%, as compared to the degree of neurodegeneration in a control (e.g., in the absence of administration of the same osteocrin-inducing agent). In one embodiment, the brain or cognitive function can be improved by more than 50%, e.g., at least about 60%, or at least about 70%, as compared to the degree of neurodegeneration in a control (e.g., in the absence of administration of the same osteocrin-inducing agent). In another embodiment, the brain or cognitive function can be improved by at least about 80%, at least about 90% or greater, as compared to the degree of neurodegeneration in a control (e.g., in the absence of administration of the same osteocrin-inducing agent).

As used herein, the term “administer” or “administration” refers to the placement of a composition comprising an osteocrin-inducing agent into a subject by a method or route which results in at least partial localization of the composition at a desired site such that desired effect is produced. Routes of administration suitable for the methods described herein include, but are not limited to, injection and/or implantation, e.g., by surgery. Generally, local administration results in more of the composition being delivered to a specific location as compared to the entire body of the subject. In some embodiments, the terms “administration”, “contacting”, and “treating” in reference to exposing neural cells to an osteocrin-inducing agent or a composition comprising the same are used interchangeably.

Increasing Neuronal Connectivity, Neuronal Survival and/or Axonal Growth of a Population of Neural Cells

The methods and/or compositions described herein can be used to increase neuronal connectivity of a population of neural cells. As used herein, the term “neuronal connectivity” generally refers to interaction and/or communication of neural cells with one another via synapses, for example, where a signal is propagated from the axon terminal or terminals located along the length of the axon of a neural cell to another neural cell's dendrite, soma (cell body), neurite, and/or axon. Synapses can be excitatory or inhibitory and either increase or decrease activity in the target neural cell. Some neural cells can also communicate via electrical synapses, which are direct, electrically-conductive junctions between cells.

By the term “increasing neuronal connectivity” or “an increase in neuronal connectivity” is meant an increase in interaction and/or communication of at least one neural cell or more (e.g., at least 2 or more neural cells) with at least one another via synapses, e.g., by at least about 10% or more, including, e.g., at least about 20%, at least about 30%, at least about 40%, at least about 50%, at least about 60%, at least about 70%, at least about 80%, at least about 90%, at least about 95%, at least about 98%, at least about 99%, or more, as compared to the neuronal connectivity of neural cells not contacted with an osteocrin-inducing agent. In some embodiments, the interaction and/or communication of at least one neural cell or more (e.g., at least 2 or more neural cells) with at least one another via synapses can be increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or more, as compared to the neuronal connectivity of neural cells not contacted with an osteocrin-inducing agent. In some embodiments, the term “increasing neuronal connectivity” or “an increase in neuronal connectivity” can refer to at least one or more neural cells having an increase in the number of dendritic branches, thus making connections with an increased number of neural cells, e.g., by at least about 10% or more, including, e.g., at least about 20%, at least about 30%, at least about 40%, at least about 50%, at least about 60%, at least about 70%, at least about 80%, at least about 90%, at least about 95%, at least about 98%, at least about 99%, or more, as compared to the number of dendritic branches and/or connections of neural cells not contacted with an osteocrin-inducing agent. Alternatively, the term “increasing neuronal connectivity” or “an increase in neuronal connectivity” can refer to at least one or more neural cells having dendritic branches making and/or receiving an increased number of synapses, e.g., by at least about 10% or more, including, e.g., at least about 20%, at least about 30%, at least about 40%, at least about 50%, at least about 60%, at least about 70%, at least about 80%, at least about 90%, at least about 95%, at least about 98%, at least about 99%, or more, as compared to the number of synapses made and/or received by neural cells not contacted with an osteocrin-inducing agent.

The neuronal connectivity of the population of neural cells can be measured and/or determined in vitro or in vivo by any methods known in the art. For example, in some embodiments of this aspect and other aspects described herein, the neuronal connectivity can be determined and/or monitored with an imaging system, e.g., a microscope, and/or a functional magnetic resonance imaging, alone or in combination with use of one or more neural markers.

In some embodiments, an increase in neuronal connectivity can be characterized and/or measured by neuronal connectivity indicators. For example, in some embodiments, an increase in neuronal connectivity can be measured by determining an increase in dendritic density in the population of neural cells by at least about 10% or more, including, e.g., at least about 20%, at least about 30%, at least about 40%, at least about 50%, at least about 60%, at least about 70%, at least about 80%, at least about 90%, at least about 95%, at least about 98%, at least about 99%, or more, as compared to the dendritic density of neural cells not contacted with an osteocrin-inducing agent. In some embodiments, the dendritic density of the population of neural cells can be increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or more, as compared to the dendritic density of neural cells not contacted with an osteocrin-inducing agent. The dendritic density can be determined, e.g., by detecting expression of at least one or more dendritic markers, e.g., but not limited to, microtubule-associated protein 2 (MAP2).

As used herein, the term “dendritic density” refers to an average number of dendritic branches that extend from a neuron cell body (soma). In some embodiments, the term “dendritic density” can encompass “dendritic spine density,” which generally refers to an average number of dendritic spines of neurons, or an average number of spine structures per unit of neuronal dendrite. Dendritic spines are generally small membranous protrusions from neurons' dendrites that typically receive input(s) form a synapse of an axon. The dendritic spines can provide an anatomical substrate for memory storage and/or synaptic transmission, and/or serve to increase the number of contacts or connections between neurons.

In some embodiments, an increase in neuronal connectivity can be measured by determining an increase in total dendritic length in the population of neural cells by at least about 10% or more, including, e.g., at least about 20%, at least about 30%, at least about 40%, at least about 50%, at least about 60%, at least about 70%, at least about 80%, at least about 90%, at least about 95%, at least about 98%, at least about 99%, or more, as compared to the total dendritic length of neural cells not contacted with an osteocrin-inducing agent. In some embodiments, the total dendritic length of the population of neural cells can be increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or more, as compared to the total dendritic length of neural cells not contacted with an osteocrin-inducing agent. The total dendritic length can be determined, e.g., by staining the dendrites of neural cells with at least one or more dendritic markers, e.g., but not limited to, microtubule-associated protein 2 (MAP2), followed by imaging (e.g., microscopy), and then measuring the length of the dendrites, e.g., using any image analysis program known in the art.

In some embodiments, an increase in neuronal connectivity can be measured by determining an increase in excitatory synapse density in the population of neural cells by at least about 10% or more, including, e.g., at least about 20%, at least about 30%, at least about 40%, at least about 50%, at least about 60%, at least about 70%, at least about 80%, at least about 90%, at least about 95%, at least about 98%, at least about 99%, or more, as compared to the excitatory synapse density of neural cells not contacted with an osteocrin-inducing agent. In some embodiments, the excitatory synapse density of the population of neural cells can be increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or more, as compared to the excitatory synapse density of neural cells not contacted with an osteocrin-inducing agent. The excitatory synapse density can be determined, e.g., by detecting expression of at least one or more synaptic markers, e.g., but not limited to, post-synaptic density (PSD) protein such as PSD95 protein, and/or synapsin. As used herein, the term “excitatory synapse density” refers a number of synapses per unit length of an excitatory neuron (e.g., an excitatory glutamatergic neuron). By way of example only, the excitatory synapse density can be calculated as the number of co-localized pre- and post-synaptic markers per unit length of an excitatory neuron (e.g., an excitatory glutamatergic neuron).

Methods to detect changes in dendritic density and/or excitatory synapse density in a population of neural cells are known in the art and can be utilized in the methods described herein. In some embodiments of this aspect and other aspects described herein, increases in the dendritic and excitatory synapse densities can be measured by determining, e.g., with immunostaining, an increased expression of at least one or more dendritic and synaptic (e.g., post-synaptic) markers, respectively. Non-limiting examples of dendritic marker and/or synaptic marker include MAP2, PSD95, synapsin, and any combinations thereof.

Additional examples of dendritic and/or synaptic markers that can be used to determine dendritic density and excitatory synaptic density, respectively, can include, without limitations, 14-3-3 eta protein, alpha-II-spectrin/alpha-Fodrin, p11 (calpactin I light chain/annexin II), ATPase Na+/K+ transporting alpha 1, adenylate cyclase III/ADCY3, alpha 2a adrenergic receptor, CADM1/synCAM, CNPase, calbindin, calretinin, ChAT, doublecortin (DCX), FOX3 (NeuN), GAD1/GAD67, GAP43, HSP105, Ki67, Lingo-1, MAP2, MARCKS (myristoylated alanine rich C kinase substrate), Mash1, matrix metalloproteinase 24 (MMP-24, MTP-MMP), myelin basic protein, NCAM-L1/CD171, NMDA NR1, NMDA receptor 1, NMDA receptor 2A, neurofilament NF, neurofilament alpha-internexin/NF66, neuron specific enolase (NSE)/ENO2, neurotensin receptor 3 (NTS3)/sortilin, PGP9.5, PLP (proteolipid protein), PTGDS, peripherin, phospho-NMDA receptor, phosphor-myelin basic protein, phosphor-synapsin, phosphor-tyrosine hydroxylase, RAGE, ROBO1, SCP1, SCP3, surviving, synapsin, synaptojanin 1, synaptophysin, synaptosomal associate protein 25, tau, tuj1 (neuron-specific class III beta-tubulin), tyrosine hydroxylase, UCHL1, VAMP1, and any combinations thereof.

In some embodiments, an increase in neuronal connectivity can be measured by determining an increase in neuronal survival in the population of neural cells. Accordingly, in one aspect, methods for increasing neuronal survival of a population of neural cells are also provided herein. The term “increase in neuronal survival” or “increasing neuronal survival” refers to an increase in the lifetime of a neural cell and/or in the number of viable neural cells under a specified condition by at least about 10% or more, including, e.g., at least about 20%, at least about 30%, at least about 40%, at least about 50%, at least about 60%, at least about 70%, at least about 80%, at least about 90%, at least about 95%, at least about 98%, at least about 99%, or more, as compared to the neuronal survival of neural cells not contacted with an osteocrin-inducing agent. In some embodiments, the lifetime of a neural cell and/or the number of viable neural cells under a specified condition can be increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or more, as compared to the neuronal survival of neural cells not contacted with an osteocrin-inducing agent. The neuronal survival of neural cells can be determined, e.g., using cell survival markers such as calcein AM, or any other art-recognized methods known in the art. In some embodiments, the neuronal survival of neural cells can be measured by determining, e.g., with immunostaining, an increased number of cells that are positive for at least one dendritic marker, e.g., but not limited to MAP2.

In some embodiments, the increase in neuronal survival of a population of neural cells contacted with an osteocrin-inducing agent is not contributed by trophic support from neighboring neural cells. As used herein, the term “trophic support” refers to trophic actions that can promote and/or control proliferation, differentiation, migration, connectivity, axonal growth and/or survival of target neurons or neural cells.

In some embodiments, an increase in neuronal connectivity can be measured by determining an increase in axonal growth of neural cells. Accordingly, in one aspect, methods for regenerating axons or increasing axonal growth of a population of neural cells are provided herein. The term “regenerating axons” or “increasing axonal growth” refers to an increase in formation and/or length of one or more long processes or axons, originating from the neuron's cell body and proceeded by the growth cone movement, by at least about 10% or more, including, e.g., at least about 20%, at least about 30%, at least about 40%, at least about 50%, at least about 60%, at least about 70%, at least about 80%, at least about 90%, at least about 95%, at least about 98%, at least about 99%, or more, as compared to the axonal growth of neural cells not contacted with an osteocrin-inducing agent. In some embodiments, the formation of one or more axons and/or an increase in the length of one or more axons of neurons or neural cells can be increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or more, as compared to the axonal growth of neural cells not contacted with an osteocrin-inducing agent. The axonal growth of neural cells can be detected by any methods known in the art. For example, in some embodiments, the axons of neural cells can be identified by the presence of at least one or more axonal markers. An exemplary axonal marker is a marker for neurofilament. Another exemplary axonal marker is a marker for Tau. Tau is a microtubule associated protein that specifically enriched in axonal microtubules. Thus, in some embodiments antibodies against neurofilament and/or Tau can be used to identify axonal features or markers of neurons. In some embodiments, the axons of neural cells do not exhibit a dendritic marker.

As used herein, the term “axon” refers to a long cellular protrusion from a neuron or neural cell, whereby action potentials or electrical impulses are conducted, either to or from the neuron's cell body.

As used herein, the term “growth cone” refers to a specialized region at the tip of a growing neurite (e.g., any protrusion from the cell body of a neuron) that is responsible for sensing the local environment and moving the axon toward its appropriate synaptic target cell.

As used herein, the term “growth cone movement” refers to the extension or collapse of the growth cone toward a neuron's target cell.

As used herein, the term “neurite” refers to any process or protrusion growing out of a cell body of a neuron. Thus, the term “neurite” can encompass a dendrite and an axon as well.

In some embodiments where the methods of various aspects described herein are performed in vivo, e.g., in a subject, an increase in neuronal connectivity, neuronal survival and/or axonal growth of neural cells can be measured by determining an increase in cognitive function (e.g., but not limited to, mental stability, memory/recall abilities, problem solving abilities, reasoning abilities, thinking abilities, judging abilities, capacity for learning, perception, intuition, awareness or any combinations thereof) of the subject by at least about 10% or more, including, e.g., at least about 20%, at least about 30%, at least about 40%, at least about 50%, at least about 60%, at least about 70%, at least about 80%, at least about 90%, at least about 95%, at least about 98%, at least about 99%, or more, as compared to the cognitive function of a subject not administered with the osteocrin-inducing agent. In some embodiments, the cognitive function (e.g., but not limited to, mental stability, memory/recall abilities, problem solving abilities, reasoning abilities, thinking abilities, judging abilities, capacity for learning, perception, intuition, awareness or any combinations thereof) of the subject can be increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or more, as compared to the cognitive function of a subject not administered with the osteocrin-inducing agent. A skilled artisan can readily perform various cognitive function tests (e.g., neuropsychological tests, and/or functional tests such as verbal tests) to assess the cognitive function of a subject.

In some embodiments where the neural cells to be treated are motor neurons or sensory neurons, one or more osteocrin-inducing agents can be administered to a subject in an amount sufficient to increase at least one or more motor and/or sensory functions (e.g., but not limited to, perception, hearing, tactile senses, locomotion, motor coordination and/or endurance, and/or vestibular function) associated with the motor neurons or sensory neurons in need thereof, an increase in neuronal connectivity, neuronal survival and/or axonal growth of motor or sensory neural cells can be measured by determining an increase in at least one motor and/or sensory functions of the subject, e.g., by at least about 10%, including, e.g., at least about 20%, at least about 30%, at least about 40%, at least about 50%, at least about 60%, at least about 70%, at least about 80%, at least about 90%, at least about 95%, at least about 98% or higher, as compared to the motor or sensory function(s) of a subject not administered with the osteocrin-inducing agent. In some embodiments, an increase in neuronal connectivity, neuronal survival and/or axonal growth of motor or sensory neural cells can be measured by determining an increase in at least one motor and/or sensory functions of the subject by at least about 1-fold, at least about 2-fold, at least about 3-fold, at least about 4-fold, at least about 5-fold, at least about 6-fold, at least about 7-fold, at least about 8-fold, at least about 9-fold, at least about 10- at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or higher, as compared to the motor or sensory function(s) of a subject not administered with the osteocrin-inducing agent. A skilled artisan can readily perform assessments of various motor/sensory functions (e.g., as part of a neurological assessment) to evaluate the motor/sensory function of a subject. For example, assessments of motor functions can include, but are not limited to, movements of upper and/or lower extremities (e.g., arm and/or leg movements). Sensory functions of a subject can be assessed, e.g., by evaluating pain and temperature sensation, position sense (proprioception) and/or light touch.

In another aspect, a method of increasing neuronal survival of a population of neural cells is also provided herein. The method comprises contacting a population of neural cells (e.g., neural cells in need of increased levels of neuronal connectivity and/or neuronal survival) with a composition comprising an effective amount of an osteocrin-inducing agent. The increase in neuronal survival of a population of neural cells contacted with an osteocrin-inducing agent is not contributed by trophic support from neighboring neural cells. In some embodiments, the population of neural cells can comprise human neural cells. In some embodiments, treatment of a population of neural cells, e.g., human neural cells, can increase neuronal survival by at least about 10% or more, including, e.g., at least about 20%, at least about 30%, at least about 40%, at least about 50%, at least about 60%, at least about 70%, at least about 80%, at least about 90%, at least about 95%, at least about 98%, at least about 99%, or more, as compared to the neuronal survival of neural cells not contacted with an osteocrin-inducing agent. In some embodiments, treatment of neural cells, e.g., human neural cells, can increase neuronal survival by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or more, as compared to the neuronal survival of neural cells not contacted with an osteocrin-inducing agent. In some embodiments, treatment of neural cells, e.g., human neural cells, can increase neuronal survival by at least about 2-fold or higher, as compared to the neuronal survival of neural cells not contacted with an osteocrin-inducing agent.

In yet another aspect, a method of regenerating axons or increasing axonal growth of a population of neural cells is also provided herein. The method comprises contacting a population of neural cells (e.g., neural cells in need of axonal growth and/or regeneration) with a composition comprising an effective amount of an osteocrin-inducing agent. In some embodiments, the population of neural cells can comprise human neural cells. In some embodiments, treatment of a population of neural cells, e.g., human neural cells, can increase axonal growth and/or regeneration by at least about 10% or more, including, e.g., at least about 20%, at least about 30%, at least about 40%, at least about 50%, at least about 60%, at least about 70%, at least about 80%, at least about 90%, at least about 95%, at least about 98%, at least about 99%, or more, as compared to the axonal growth and/or regeneration of neural cells not contacted with an osteocrin-inducing agent. In some embodiments, treatment of neural cells, e.g., human neural cells, can increase axonal growth and/or regeneration by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or more, as compared to the axonal growth and/or regeneration of neural cells not contacted with an osteocrin-inducing agent. In some embodiments, treatment of neural cells, e.g., human neural cells, can increase axonal growth and/or regeneration by at least about 2-fold or higher, including, e.g., at least about 3-fold, at least about 4-fold, or higher, as compared to the axonal growth and/or regeneration of neural cells not contacted with an osteocrin-inducing agent.

### Osteocrin-Inducing Agent and Effective Amount Thereof

Osteocrin (OSTN) is known as a secreted protein produced by cells of osteoblast lineage. OSTN can modulate osteoblast phenotype and can be used to stimulate bone growth and/or treatment of osteoporosis. See, e.g., Thomas et al. (2003) Journal of Biological Chemistry 278: 50563-50571; Moffatt et al. (2007) Journal of Biological Chemistry 282: 36454-36462; and U.S. Pat. No. 7,479,668, the content of which is incorporated herein by reference. However, there are no reports on roles of osteocrin in nervous system development or particularly human nervous system development. The inventors have surprisingly discovered inter alia that osteocrin is a human-specific neural activity-regulated gene where the OSTN levels were unaffected in non-human neural cells, e.g., mouse neural cells. Further, the inventors have discovered that OSTN can increase human neuronal connectivity, e.g., increased excitatory synapse density by at least about 3-fold or higher, as compared to neural cells without treatment of OSTN. The OSTN can also increase human neuronal survival and/or axonal growth of human neurons, e.g., by at least about 2-fold or higher, as compared to neural cells without treatment of OSTN.

In accordance with embodiments of some aspects described herein, an effective amount of an osteocrin-inducing agent is contacted with a population of neural cells to increase neuronal activity, neural survival and/or axonal growth of the population of neural cells. In some embodiments, an effective amount of an osteocrin-inducing agent can be contacted with a population of neural cells in vitro or ex vivo. In some embodiments, an effective amount of an osteocrin-inducing agent can be administered in vivo to a subject (e.g., a human subject). Ex vivo treatment of neural cells with an osteocrin-inducing agent, followed by re-introduction of the cells into a subject, or in vivo administration of an osteocrin-inducing agent can be used to treat and/or prevent a neurodegenerative condition in a subject, and/or to promote axonal growth and/or regeneration in a subject.

An osteocrin-inducing agent is any agent that can directly or indirectly increase intracellular and/or extracellular (secreted) levels of osteocrin by at least about 5%, including, for example, at least about 10%, at least about 20%, at least about 30%, at least about 40%, at least about 50%, at least about 60%, at least about 70%, at least about 80%, at least about 90%, at least about 95%, at least about 98%, or more. In one embodiment, an osteocrin-inducing agent is an agent that can directly or indirectly increase intracellular and/or extracellular (secreted) levels of human osteocrin, e.g., by at least about 5% (including, e.g., at least about 10% or more) within a population of neural cells. Examples of an osteocrin-inducing agent can include, but are not limited to, a recombinant osteocrin protein or a portion thereof (e.g., a peptide comprising C-terminus of osteocrin) or a peptidomimetic thereof, a recombinant osteocrin-encoding gene, optionally operably linked to an expression vector (e.g., a viral vector and/or a plasmid vector), a small molecule that induces intracellular and/or extracellular (secreted) expression of osteocrin, a small-molecule osteocrin analog or a prodrug thereof, a neural cell (e.g., a neural stem cell) engineered with enhanced intracellular and/or extracellular (secreted) expression of osteocrin, and any combinations thereof.

Osteocrin-Inducing Proteins and Peptides:

In some embodiments, an osteocrin-inducing agent can comprise an osteocrin protein or a portion thereof, or a peptidomimetic thereof. An osteocrin protein, for example, can be isolated and purified from osteocrin-producing cells, e.g., cells of osteoblast lineage, or from any tissues where these cells are present, for example, bone tissues, skeletal muscles, or brain tissue of mammals (for example, humans, mice, rats, rabbits, sheep, swine, cattle, horses, cats, dogs, monkeys, chimpanzee). Alternatively, the osteocrin protein can also be a chemically synthesized protein or a protein biochemically synthesized using a cell-free translation system, or a recombinant protein produced from a transformant incorporating a nucleic acid having a nucleotide sequence encoding an amino acid sequence of osteocrin or a portion thereof.

In some embodiments, the osteocrin-inducing agent can comprise a recombinant osteocrin protein or peptide. In some embodiments, the osteocrin-inducing agent can comprise an amino acid sequence of at least a portion of human osteocrin and display an ability to promote neuronal connectivity, neuronal survival and/or axonal growth of human neural cells. In some embodiments, the osteocrin-inducing agent can comprise an amino acid sequencing having at least about 70% or more homology (including, at least about 80%, at least about 90%, at least about 95%, at least about 98% or more homology) to at least a portion of human osteocrin and display an ability to promote neuronal connectivity, neuronal survival and/or axonal growth of human neural cells. As used herein, the term “homology” refers to the proportion (%) of the same or similar amino acid residues to all overlapping amino acid residues in an optimal alignment where two amino acid sequences are aligned using a mathematic algorithm known in the art. For example, amino acid sequence homology can be calculated using the homology calculation algorithm NCBI BLAST (National Center for Biotechnology Information Basic Local Alignment Search Tool) or any other sequence alignment algorithms known in the art. The term “similar amino acid residues” refers to amino acid residues having similar physiochemical properties; for examples, amino acids classified under the same group, such as aromatic amino acids (Phe, Trp, Tyr), aliphatic amino acids (Ala, Leu, He, Val), polar amino acids (Gin, Asn), basic amino acids (Lys, Arg, His), acidic amino acids (Glu, Asp), amino acids having a hydroxy group (Ser, Thr), and amino acids having a small side chain (Gly, Ala, Ser, Thr, Met). Similar amino acid residues also encompass amino acids suitable for conservative amino acid substitution.

The amino acid sequences of human osteocrin are known in the art. An exemplary amino sequence of human osteocrin is indicated by SEQ ID NO: 2. In some embodiments, the osteocrin-inducing agent can comprise an amino acid sequence with a homology of at least about 70% or more, including, e.g., at least about 80% or more, at least about 90% or more, and at least about 95% or more, to at least a functional portion of the amino acid sequence shown by SEQ ID NO. 2.

As used herein, the term “a functional portion” refers to a portion of an amino acid sequence or a nucleotide sequence that controls neuronal connectivity, neuronal survival and/or axonal growth of neural cells. Other amino acid sequences of human osteocrin or other species, which differ from the corresponding naturally occurring osteocrin, e.g., by conservative substitution (e.g., substitution by similar amino acid residues), can also be used to generate an osteocrin-inducing agent.

In various embodiments, the structure of an osteocrin-inducing agent can be modified for such purposes as enhancing therapeutic efficacy, or stability (e.g., ex vivo shelf life or resistance to proteolytic degradation in vivo). Modified osteocrin-inducing agents can be produced, for instance, by amino acid substitution, deletion, or addition. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of an osteocrin-inducing agent results in a functional homolog can be readily determined by assessing the ability of the variant osteocrin-inducing peptide to produce a response (e.g., measurement of neuronal connectivity, neuronal survival and/or axonal growth as described herein and in the Examples) in neural cells in a fashion similar to the wild-type osteocrin-inducing protein.

In some embodiments, an osteocrin-inducing agent can comprise an osteocrin peptide. A peptide can be a fragment of the naturally occurring protein, or a mimetic or peptidomimetic of osteocrin. Variants of osteocrin-inducing peptides can be generated by mutagenesis (e.g., amino acid substitution, amino acid insertion, or truncation), and identified by screening combinatorial libraries of mutants, such as truncation mutants, for the desired activity, e.g., increased neuronal connectivity. In some embodiments, an osteocrin-inducing peptide can comprise a portion of a human osteocrin protein, e.g., a peptide comprising C-terminus of human osteocrin (e.g., a peptide comprising C-terminus of human osteocrin, wherein the amino acid sequence of human osteocrin is indicated by SEQ ID NO: 2). In some embodiments, an osteocrin-inducing peptide can comprise a portion of a human osteocrin amino acid sequence with homology to the natriuretic peptides (NPs).

The osteocrin-inducing peptide can be produced by recombinant methods or direct chemical synthesis. Further, the peptide can be produced as a modified peptide, with nonpeptide moieties attached, e.g., by covalent linkage, to the N-terminus and/or C-terminus. In some embodiments, either the carboxy-terminus or the amino-terminus, or both, can be chemically modified. Some common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments described herein. Certain amino-terminal and/or carboxy-terminal modifications and/or peptide extensions to the core sequence can change physical, chemical, biochemical, and pharmacological properties, such as: increased nuclear import, enhanced stability, cell permeability, increased potency and/or efficacy, resistance to serum proteases, and desirable pharmacokinetic properties.

As used herein, the term “peptidomimetic” means a peptide-like molecule that has the activity of the peptide on which it is structurally based. Such peptidomimetics include chemically modified peptides, peptide-like molecules containing non-naturally occurring amino acids, and peptoids, and have an activity of the peptide upon which the peptidomimetic is derived (see, for example, Goodman and Ro, Peptidomimetics for Drug Design, in “Burger's Medicinal Chemistry and Drug Discovery”, Vol. 1 (ed. M. E. Wolff; John Wiley & Sons 1995), pages 803-861). A variety of peptidomimetics are known in the art and can be encompassed within embodiments described herein including, for example, peptide-like molecules which contain a constrained amino acid, a non-peptide component that mimics peptide secondary structure, or an amide bond isostere. A peptidomimetic that contains a constrained, non-naturally occurring amino acid can include, for example, an α-methylated amino acid; α,α-dialkylglycine or α-aminocycloalkane carboxylic acid; an Nα-Cαcyclized amino acid; an Nα-methylated amino acid; αβ, or γ-amino cycloalkane carboxylic acid; an α,β-unsaturated amino acid; a β,β-dimethyl or β-methyl amino acid; αβ-substituted-2,3-methano amino acid; an N-Cδ or Cα-Cδ cyclized amino acid; a substituted proline or another amino acid mimetic. A peptidomimetic which mimics peptide secondary structure can contain, for example, a nonpeptidic β-turn mimic; γ-turn mimic; mimic of β-sheet structure; or mimic of helical structure, each of which is well known in the art. A peptidomimetic also can be a peptide-like molecule which contains, for example, an amide bond isostere such as a retro-inverso modification; reduced amide bond; methylenethioether or methylene-sulfoxide bond; methylene ether bond; ethylene bond; thioamide bond; transolefin or fluoroolefin bond; 1,5-disubstituted tetrazole ring; ketomethylene or fluoroketomethylene bond or another amide isostere. One skilled in the art understands that these and other peptidomimetics are encompassed within the meaning of the term “peptidomimetic” as used herein.

Methods for identifying a peptidomimetic are well known in the art and include, for example, the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). This structural depository is continually updated as new crystal structures are determined and can be screened for compounds having suitable shapes, for example, the same shape as a peptide described herein, as well as potential geometrical and chemical complementarity to a cognate receptor. Where no crystal structure of a peptide described herein is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of a peptide described herein, for example, having specificity for the microbes.

In some embodiments, an osteocrin-inducing peptide or protein can be a recombinant osteocrin. In some embodiments, the recombinant osteocrin can comprise an N-terminal and/or C-terminal label. For example, the N-terminal and/or C-terminal label can be a His-tag. Methods to produce recombinant proteins and/or peptides are known in the art, e.g., but not limited to, production by E. coli, and can be used to produce a recombinant osteocrin. In some embodiments, the recombinant osteocrin can comprise an N-terminal label, e.g., but not limited to, a His-tag.

In some embodiments, the recombinant osteocrin or an osteocrin-inducing agent can have an amino acid sequence of no more than 1000 amino acid residues, no more than 750 amino acid residues, no more than 500 amino acid residues, no more than 250 amino acid residues, no more than 200 amino acid residues, no more than 150 amino acid residues, no more than 125 amino acid residues. In some embodiments, the recombinant osteocrin or an osteocrin-inducing agent can have an amino acid sequence having a length of about 50 amino acid residues to about 500 amino acid residues, or about 75 amino acid residues to about 250 amino acid residues, or about 100 amino acid residues to about 150 amino acid residues. In some embodiments, the recombinant osteocrin or an osteocrin-inducing agent can have an amino acid sequence having a length of about 75 amino acid residues to about 150 amino acid residues.

In some embodiments, the recombinant osteocrin or an osteocrin-inducing agent can have a molecular weight of about 0.5 kDa to about 100 kDa, or about 1 kDa to about 50 kDa, or about 5 kDa to about 25 kDa, or about 10 kDa to about 20 kDa, or about 10 kDa to about 15 kDa. In some embodiments, the recombinant osteocrin or an osteocrin-inducing agent can have a molecular weight of about 5 kDa to about 15 kDa.

In some embodiments, the recombinant osteocrin or an osteocrin-inducing agent can comprise an enzyme cleavage site. Without wishing to be limiting, in some embodiments, the recombinant osteocrin or an osteocrin-inducing agent can comprise a Furin cleavage site (e.g., but not limited to, a short amino acid sequence comprising TK). In these embodiments where the recombinant osteocrin or an osteocrin-inducing agent comprises an enzyme cleavage site, the recombinant osteocrin or the osteocrin-inducing agent can be cleaved to produce a smaller N-terminal osteocrin peptide or C-terminal osteocrin peptide.

In some embodiments, the recombinant osteocrin can comprise an amino acid sequence (having an N-terminal His-tag) as follows:

### MRGSHHHHHH GMASHMVDVT TTEAFDSGVI DVQSTPTVRE EKSATDLTAK LLLLDELVSL ENDVIETKKK RSFSGFGSPL DRLSAGSVDH KGKQRKVVDH PKRRFGIPMD RIGRNRLSNS RG (SEQ ID NO:3)

where the amino acid residues in bold correspond to a His-Tag or they can be replaced by another art-recognized label. The underlined amino acid residues can correspond to a Furin cleavage site or be replaced by other enzyme cleavage site known in the art. In this embodiment, when the cleavage occurs, it would create a ˜6 kDa OSTN C-terminal peptide.

In some embodiments, the osteocrin-inducing agent as described herein, e.g., osteocrin-inducing protein or peptide, can be modified to increase its stability. For example, in some embodiments, at least one polyethylene glycol (PEG) moiety can be attached to an osteocrin-inducing protein or peptide, e.g., to increase the stability of the recombinant protein. PEGylation is a common approach in drug delivery and applicable to the size of osteocrin.

Osteocrin-Inducing Small Molecules:

In some embodiments, an osteocrin-inducing agent can comprise a small molecule that can directly or indirectly induce intracellular and/or extracellular (secreted) expression of osteocrin, e.g., human osteocrin. In some embodiments, the small molecule that can directly or indirectly induce intracellular and/or extracellular expression of osteocrin, e.g., human osteocrin, but are not known for treatment of a neurodegenerative and/or neurological disorder and/or condition, e.g., Alzheimer's disease. In one embodiment, the small molecule excludes a peroxisome proliferator-activated receptor gamma (PPAR-gamma) agonist, e.g., Troglitazone.

In some embodiments, the small molecule can comprise an osteocrin analog, e.g., an osteocrin mimimetics that can interact with a cell receptor to which an osteocrin protein generally binds, or a prodrug thereof. For example, the small molecule can comprise a ligand or an agonist for a natriuretic peptide clearance receptor.

Osteocrin-inducing small molecules can be obtained using any of the numerous approaches in combinatorial library methods known in the art, including, e.g., biological libraries; spatially addressable parallel solid phase or solution phase libraries; synthetic library methods requiring deconvolution; the ‘one-bead one-compound’ library method; and synthetic library methods using affinity chromatography selection. The biological library approach can be applied to peptide libraries, while the other four approaches are applicable to peptide, non-peptide oligomer or small molecule libraries of compounds. Libraries of compounds can be presented in solution, and/or on beads, chips, bacteria, spores, plasmids, and/or phage.

Examples of methods for synthesis of molecular libraries are known in the art, for example in: U.S. Pat. App. No.: US 2005/0079540 entitled “Method for conducting solid phase synthesis of molecule libraries using combinatorial sealing matrices;” and U.S. Pat. Nos.: U.S. Pat. No. 8,168,381 entitled “Template directed split and mix synthesis of small molecule libraries;” U.S. Pat. No. 7,972,992 entitled “Methods for synthesis of encoded libraries;” U.S. Pat. No. 7,718,578 entitled “Method of synthesis and testing of combinatorial libraries using microcapsules;” and U.S. Pat. No. 6,090,912 entitled “Topologically segregated, encoded solid phase libraries comprising linkers having an enzymatically susceptible bond.”

In some embodiments, one or more active motifs of osteocrin protein can be identified using art-recognized methods to design and/or screen chemicals that mimic the osteocrin's effect on neurons.

Osteocrin-Inducing Nucleic Acid Molecules:

In some embodiments, the osteocrin-inducing agent can be a nucleic acid molecule, for example, a nucleic acid molecule comprising a nucleotide sequence of osteocrin or a functional portion thereof (e.g., a portion that controls neural connectivity, neural survival and/or axonal growth of neurons). In some embodiments, the osteocrin-inducing agent can comprise a nucleotide sequence of human osteocrin (as indicated by SEQ ID NO. 1) or a functional portion thereof. Other nucleotide sequences of human osteocrin or osteocrin from other species, including the ones which differ from the corresponding naturally occurring nucleotide sequences in codon sequence due to the degeneracy of the genetic code, can also be used to generate an osteocrin-inducing nucleic acid molecule.

The osteocrin-inducing nucleic acid molecules can be delivered to target neural cells by transfection, thus increasing the intracellular and/or extracellular (secreted) expression of osteocrin. Accordingly, in some embodiments, the osteocrin-inducing agent can comprise a recombinant osteocrin-encoding gene or a portion thereof.

Given the sequences encoding osteocrin of various species (e.g., but not limited to, human, mouse, rat, and cow) are disclosed in the art, an osteocrin-inducing nucleic acid molecule for use in the methods described herein can be constructed using chemical synthesis and enzymatic ligation reactions using procedures known in the art. For example, a nucleic acid molecule can be chemically or recombinantly synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

In yet another embodiment, the osteocrin-inducing nucleic acid molecules can be modified at the base moiety, sugar moiety, or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup and Nielsen (1996) supra and Perry-O'Keefe et al. (1996) Proc. Natl. Acad. Sci. USA 93:14670-675.

In some embodiments, an expression vector (e.g., but not limited to a viral vector) can be used to deliver osteocrin-inducing nucleic acid molecules into target neural cells for inducing intracellular and/or extracellular (secreted) expression of osteocrin or a portion thereof. Hence, in some embodiments, the osteocrin-inducing nucleic acid molecules (e.g., recombinant osteocrin-encoding gene) can be operably linked to an expression vector (e.g., but not limited to, a viral vector, or a plasmid vector). Some viral-mediated expression methods employ retrovirus, adenovirus, lentivirus, herpes virus, pox virus, and adeno-associated virus (AAV) vectors, and such expression methods have been used in gene delivery and are well known in the art. For example, U.S. patent application No. 2002/0,193,335 provides methods of delivering a gene therapy vector, or transformed cell, to neurological tissue; U.S. patent application No. 2002/0,187,951 provides methods for treating or preventing a neurodegenerative disease in a mammal by administering a lentiviral vector to a target cell in the brain or nervous system of the mammal; U.S. patent application No. 2002/0,107,213 discloses a gene therapy vehicle and methods for its use in the treatment and prevention of neurodegenerative disease; U.S. patent application No. 2003/0,099,671 discloses a mutated rabies virus suitable for delivering a gene to a subject; and U.S. Pat. No. 6,310,196 describes a DNA construct which is useful for immunization or gene therapy; U.S. Pat. No. 6,436,708 discloses a gene delivery system which results in long-term expression throughout the brain has been developed; U.S. Pat. No. 6,140,111 which disclose retroviral vectors suitable for human gene therapy in the treatment of a variety of disease; and Kaspar B K et al. (2002) Mol Ther. 5:50-6, Suhr S T et al (1999) Arch Neurol. 56:287-92, Wong, P. C. et al. (2002) Nat Neurosci 5, 633-639) describes neuronal specific promoters such as Thy1 which can be employed in the methods described herein.

Retroviral Gene Delivery:

Retroviruses provide a convenient platform for gene delivery. A selected gene can be inserted into a vector and packaged in retroviral particles using techniques known in the art. The recombinant virus can then be isolated and delivered to target cells of the subject either in vivo or ex vivo. A number of retroviral systems have been described. See, e.g., U.S. Pat. No. 5,219,740; Miller and Rosman (1989) BioTechniques 7:980-90; Miller, A. D. (1990) Human Gene Therapy 1:5-14; Scarpa et al. (1991) Virology 180:849-52; Burns et al. (1993) Proc. Natl. Acad. Sci. USA 90:8033-37; Boris-Lawrie and Temin (1993) Curr. Opin. Genet. Develop. 3:102-09.

Replication-defective murine retroviral vectors are widely used gene transfer vectors. Murine leukemia retroviruses include a single stranded RNA molecule complexed with a nuclear core protein and polymerase (pol) enzymes, encased by a protein core (gag), and surrounded by a glycoprotein envelope (env) that determines host range. The genomic structure of retroviruses includes gag, pol, and env genes and 5′ and 3′ long terminal repeats (LTRs). Retroviral vector systems exploit the fact that a minimal vector containing the 5′ and 3′ LTRs and the packaging signal are sufficient to allow vector packaging, infection and integration into target cells, provided that the viral structural proteins are supplied in trans in the packaging cell line. Fundamental advantages of retroviral vectors for gene transfer include efficient infection and gene expression in most cell types, precise single copy vector integration into target cell chromosomal DNA and ease of manipulation of the retroviral genome.

Adenoviral Delivery:

In one embodiment, a nucleotide sequence encoding an osteocrin-inducing agent can be inserted into an adenovirus-based expression vector. Unlike retroviruses, which integrate into the host genome, adenoviruses persist extrachromosomally thus minimizing the risks associated with insertional mutagenesis (Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76).

The adenovirus genome is a linear double-stranded DNA molecule of approximately 36,000 base pairs with the 55-kDa terminal protein covalently bound to the 5′ terminus of each strand. Adenoviral (“Ad”) DNA contains identical Inverted Terminal Repeats (“ITRs”) of about 100 base pairs with the exact length depending on the serotype. The viral origins of replication are located within the ITRs exactly at the genome ends.

Adenoviral vectors have several advantages in gene therapy. They infect a wide variety of cells, have a broad host-range, exhibit high efficiencies of infectivity, direct expression of heterologous genes at high levels, and achieve long-term expression of those genes in vivo. The virus is fully infective as a cell-free virion so injection of producer cell lines is not necessary. With regard to safety, adenovirus is not associated with severe human pathology, and the recombinant vectors derived from the virus can be rendered replication defective by deletions in the early-region 1 (“E1”) of the viral genome. Adenovirus can also be produced in large quantities with relative ease. For all these reasons vectors derived from human adenoviruses, in which at least the E1 region has been deleted and replaced by a gene of interest, have been used extensively for gene therapy experiments in the pre-clinical and clinical phase.

Adenoviral vectors for use in the methods described herein can be derived from any of the various adenoviral serotypes, including, without limitation, any of the over 40 serotype strains of adenovirus, such as serotypes 2, 5, 12, 40, and 41. The adenoviral vectors used herein are replication-deficient and contain the gene of interest under the control of a suitable promoter, such as any of the promoters discussed below with reference to adeno-associated virus. For example, U.S. Pat. No. 6,048,551, incorporated herein by reference, describes replication-deficient adenoviral vectors that can be used to include a Osteocrin-inducing agent under the control of the Rous Sarcoma Virus (RSV) promoter.

Other recombinant adenoviruses of various serotypes, and comprising different promoter systems, can be created by those skilled in the art. See, e.g., U.S. Pat. No. 6,306,652, incorporated herein by reference.

Moreover, “minimal” adenovirus vectors as described in U.S. Pat. No. 6,306,652 can be used to deliver osteocrin-inducing nucleic acid molecules described herein. Such vectors retain at least a portion of the viral genome required for encapsidation (the encapsidation signal), as well as at least one copy of at least a functional part or a derivative of the ITR. Packaging of the minimal adenovirus vector can be achieved by co-infection with a helper virus or, alternatively, with a packaging-deficient replicating helper system.

Other useful adenovirus-based vectors for delivery of an osteocrin-inducing agent include the “gutless” (helper-dependent) adenovirus in which the vast majority of the viral genome has been removed. Wu et al. (2001) Anesthes. 94:1119-32. Such “gutless” adenoviral vectors produce essentially no viral proteins, thus allowing gene therapy to persist for over a year after a single administration. Parks (2000) Clin. Genet. 58:1-11; Tsai et al. (2000) Curr. Opin. Mol. Ther. 2:515-23. In addition, removal of the viral genome creates space that can be used to insert control sequences that provide for regulation of transgene expression by systemically administered drugs (Burcin et al. (1999) Proc. Natl. Acad. Sci. USA 96:355-60), adding both safety and control of virally driven protein expression. These and other recombinant adenoviruses can also be used in the methods described herein.

Adeno Associated Virus (AAV):

One viral system that has been used for gene delivery is AAV. AAV is a parvovirus which belongs to the genus Dependovirus. AAV has several attractive features not found in other viruses. First, AAV can infect a wide range of host cells, including non-dividing cells. Second, AAV can infect cells from different species. Third, AAV has not been associated with any human or animal disease and does not appear to alter the biological properties of the host cell upon integration. Indeed, it is estimated that 80-85% of the human population has been exposed to the virus. Finally, AAV is stable at a wide range of physical and chemical conditions, facilitating production, storage and transportation.

The AAV genome is a linear single-stranded DNA molecule containing approximately 4681 nucleotides. The AAV genome generally comprises an internal nonrepeating genome flanked on each end by inverted terminal repeats (ITRs). The ITRs are approximately 145 base pairs (bp) in length. The ITRs have multiple functions, including serving as origins of DNA replication and as packaging signals for the viral genome.

The internal non-repeated portion of the genome includes two large open reading frames, known as the AAV replication (rep) and capsid (cap) genes. The rep and cap genes code for viral proteins that allow the virus to replicate and package the viral genome into a virion. In particular, a family of at least four viral proteins is expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus; that is, it requires co-infection with a helper virus (e.g., adenovirus, herpes virus or vaccinia) in order to form AAV virions in the wild. In the absence of co-infection with a helper virus, AAV establishes a latent state in which the viral genome inserts into a host cell chromosome, but infectious virions are not produced. Subsequent infection by a helper virus rescues the integrated genome, allowing it to replicate and package its genome into infectious AAV virions. While AAV can infect cells from different species, the helper virus must be of the same species as the host cell. Thus, for example, human AAV will replicate in canine cells co-infected with a canine adenovirus.

Adeno-associated virus (AAV) has been used with success in gene therapy. AAV has been engineered to deliver genes of interest by deleting the internal nonrepeating portion of the AAV genome (i.e., the rep and cap genes) and inserting a heterologous gene (in this case, the gene encoding the anti-inflammatory cytokine) between the ITRs. The heterologous gene is typically functionally linked to a heterologous promoter (constitutive, cell-specific, or inducible) capable of driving gene expression in the patient's target cells under appropriate conditions.

Recombinant AAV virions comprising an osteocrin-inducing nucleic acid molecule can be produced using a variety of art-recognized techniques. In one embodiment, a rAAV vector construct is packaged into rAAV virions in cells co-transfected with wild-type AAV and a helper virus, such as adenovirus. See, e.g., U.S. Pat. No. 5,139,941.

Alternatively, plasmids can be used to supply the necessary replicative functions from AAV and/or a helper virus. In one embodiment, rAAV virions are produced using a plasmid to supply necessary AAV replicative functions (the “AAV helper functions”). See e.g., U.S. Pat. Nos. 5,622,856 and 5,139,941, both incorporated herein by reference in their entireties. In another embodiment, a triple transfection method is used to produce rAAV virions. The triple transfection method is described in detail in U.S. Pat. Nos. 6,001,650 and 6,004,797, which are incorporated by reference herein in their entireties. The triple transduction method is advantageous because it does not require the use of an infectious helper virus during rAAV production, enabling production of a stock of rAAV virions essentially free of contaminating helper virus. This is accomplished by use of three vectors for rAAV virion production: an AAV helper function vector, an accessory function vector, and a rAAV expression vector. One of skill in the art will appreciate, however, that the nucleic acid sequences encoded by these vectors can be provided on two or more vectors in various combinations. Vectors and cell lines necessary for preparing helper virus-free rAAV stocks are commercially available as the AAV Helper-Free System (Catalog No. 240071) (Stratagene, La Jolla, Calif.).

The AAV helper function vector encodes AAV helper function sequences (i.e., rep and cap) that function in trans for productive rAAV replication and encapsidation. Preferably, the AAV helper function vector supports efficient rAAV virion production without generating any detectable replication competent AAV virions (i.e., AAV virions containing functional rep and cap genes). An example of such a vector, pHLP19, is described in U.S. Pat. No. 6,001,650. The rep and cap genes of the AAV helper function vector can be derived from any of the known AAV serotypes. For example, the AAV helper function vector may have a rep gene derived from AAV-2 and a cap gene derived from AAV-6. One of skill in the art will recognize that other rep and cap gene combinations are possible, the defining feature being the ability to support rAAV virion production.

The accessory function vector encodes nucleotide sequences for non-AAV-derived viral and/or cellular functions upon which AAV is dependent for replication (the “accessory functions”). The accessory functions include those functions required for AAV replication, including, without limitation, genes involved in activation of AAV gene transcription, stage specific AAV mRNA splicing, AAV DNA replication, synthesis of cap expression products, and AAV capsid assembly. Viral-based accessory functions can be derived from any of the well-known helper viruses such as adenovirus, herpes virus (other than herpes simplex virus type-1), and vaccinia virus. In one embodiment, the accessory function plasmid pLadeno5 can be used. See U.S. Pat. No. 6,004,797. This plasmid provides a complete set of adenovirus accessory functions for AAV vector production, but lacks the components necessary to form replication-competent adenovirus.

Unlike stocks of rAAV vectors prepared using infectious helper virus, stocks prepared using an accessory function vector (e.g. the triple transfection method) do not contain contaminating helper virus because no helper virus is added during rAAV production. Even after purification, for example by CsCl density gradient centrifugation, rAAV stocks prepared using helper virus still remain contaminated with some level of residual helper virus. When adenovirus is used as the helper virus in preparing a stock of rAAV virions, contaminating adenovirus can be inactivated by heating to temperatures of approximately 60° C. for 20 minutes or more. This treatment effectively inactivates only the helper virus since AAV is extremely heat stable, while the helper adenovirus is heat labile. Although heat inactivating of rAAV stocks may render much of the contaminating adenovirus non-infectious, it does not physically remove the helper virus proteins from the stock. Such contaminating viral protein can elicit undesired immune responses in subjects and are to be avoided if possible. Contaminating adenovirus particles and proteins in rAAV stocks can be avoided by use of the accessory function vectors disclosed herein.

Recombinant AAV Expression Vectors:

Recombinant AAV expression vectors can be constructed using standard techniques of molecular biology. rAAV vectors comprise a transgene of interest (e.g. a sequence comprising a human osteocrin gene (SEQ ID NO. 1) or a portion thereof) flanked by AAV ITRs at both ends. rAAV vectors are also constructed to contain transcription control elements operably linked to the transgene sequence, including a transcriptional initiation region and a transcriptional termination region. The control elements are selected to be functional in a mammalian target cell.

The nucleotide sequences of AAV ITR regions are known. See, e.g., Kotin (1994) Human Gene Therapy 5:793-801; Berns “Parvoviridae and their Replication” in Fundamental Virology, 2nd Edition, (B. N. Fields and D. M. Knipe, eds.) for the AAV-2 sequence. AAV ITRs used in the vectors need not have a wild-type nucleotide sequence, and may be altered, e.g., by the insertion, deletion or substitution of nucleotides. Additionally, AAV ITRs may be derived from any of several AAV serotypes, including without limitation, AAV-1, AAV-2, AAV-3, AAV-4, AAV-5, AAV-6, AAV-7 and AAV-8, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

Suitable transgenes for delivery in AAV vectors can be less than about 5 kilobases (kb) in size. In one embodiment, an osteocrin-inducing nucleic acid molecule comprising a nucleotide sequence of SEQ ID NO. 1 can be delivered with AAV vectors. The selected polynucleotide sequence is operably linked to control elements that direct the transcription thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, neuron-specific enolase promoter, a GFAP promoter, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, Calif.).

The AAV expression vector harboring a transgene of interest bounded by AAV ITRs can be constructed by directly inserting the selected sequence(s) into an AAV genome that has had the major AAV open reading frames (“ORFs”) excised. Other portions of the AAV genome can also be deleted, so long as enough of the ITRs remain to provide replication and packaging functions. Such constructs can be designed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-96; Vincent et al. (1990) Vaccines 90 (Cold Spring Harbor Laboratory Press); Carter (1992) Current Opinion in Biotechnology 3:533-39; Muzyczka (1992) Current Topics in Microbiol. and Immunol. 158:97-129; Kotin (1994) Human Gene Therapy 5:793-801; Shelling and Smith (1994) Gene Therapy 1:165-69; and Zhou et al. (1994) J. Exp. Med. 179:1867-75.

AAV ITR-containing DNA fragments can be ligated at both ends of a selected transgene using standard techniques, such as those described in Sambrook et al., supra. For example, ligations can be accomplished in 20 mM Tris-Cl pH 7.5, 10 mM MgCl2, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration).

Suitable host cells for producing rAAV virions from rAAV expression vectors include microorganisms, yeast cells, insect cells, and mammalian cells. Such host cells are preferably capable of growth in suspension culture, a bioreactor, or the like. The term “host cell” includes the progeny of the original cell that has been transfected with a rAAV virion. Cells from the stable human cell line, 293 (readily available through the American Type Culture Collection under Accession Number ATCC CRL1573) can be used in the practice of various embodiments described herein. The human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a particularly convenient platform in which to produce rAAV virions.

While osteocrin-inducing nucleic acid molecules can be delivered to neural cells in situ, e.g., degenerating neural cells, in a subject, e.g., using AAV expression vectors, to induce expression of secreted osteocrin, in some embodiments, neural cells (e.g., autologous or allogeneic neural cells or neural stem cells) can be engineered for enhanced intracellular and/or extracellular (secreted) expression of osteocrin, e.g., by transfecting or transducing them with expression vectors operably linked to a recombinant osteocrin-encoding gene, prior to implantation into a target site, e.g., of a brain, in a subject.

Since osteocrin is a human-specific neural activity-regulated gene, where induction of osteocrin expression by neuronal activity and/or depolarization occurs selectively in human neurons (not in, e.g., mouse neurons), in some embodiments, an osteocrin-inducing agent for use in the methods and/or compositions described herein can comprise an agent or molecule that has been developed or discovered in non-human animal models (e.g., but not limited to, mouse or rat) for increasing osteocrin expression in non-neurons and/or non-brain tissues (e.g., but not limited to, bone or skeletal tissues). In some embodiments, an osteocrin-inducing agent for use in the methods and/or compositions described herein can comprise an osteocrin-inducing agent or molecule that has been developed in non-human animal models (e.g., but not limited to, mouse or rat) for treatment of non-neuronal diseases or disorders (e.g., but not limited to, bone diseases or disorders). Accordingly, in some embodiments, the methods and/or compositions described herein can represent a treatment that is tailored to features of neurons that are uniquely human and therefore have not previously been targeted by osteocrin-inducing agents that have been developed using non-human animal models (e.g., but not limited to mouse or rat).

Additional examples of an osteocrin-inducing agent for use in the methods and/or compositions described herein can include, but are not limited to, ligands of the natriuretic clearance receptor (NPR-C) that block the clearance action of NPR-C to locally elevate levels of the natriuretic peptides (including, e.g., but not limited to, naturally-occurring and/or synthetic OSTN, OSTN fusion protein, and NPR-C specific OSTN peptide derivatives with amino acid or nucleotide sequences disclosed in U.S. Pat. No. 7,470,668), chimerae comprising an N-terminal OSTN fragment (including, e.g., but not limited to, the osteocrin “NPR-C inhibitor” fragment-CNP22 chimera with the amino acid sequence disclosed in U.S. Pat. App. No. US 2010/0331256), musclin protein (including, e.g., but not limited to, musclin protein disclosed in U.S. Pat. No. 2011/0104705 and the corresponding sequences in other species, e.g., human), and any combinations thereof. The contents of the cited U.S. patent and patent applications are incorporated herein by reference.

In some embodiments, an osteocrin-inducing agent can comprise an insulin and/or IGF-1.

In some embodiments, an osteocrin-inducing agent can be an agent that can induce depolarization in a cell. In some embodiments, the agent can be a small molecule, a nucleic acid molecule, a peptide, a protein, an aptamer, a physical, chemical, biological, mechanical and/or electrical stimulation, or any combinations thereof.

Osteocrin is also known as musclin. In some embodiments, osteocrin/musclin can be expressed and secreted from muscle cells and used in treatment of motor neurons and regulate different aspects of the neuronal functions at the neuromuscular synapses.

In embodiments of the methods described herein, a population of neural cells is contacted with an effective amount of at least one or more (e.g., 1, 2, 3, 4 or more) osteocrin-inducing agent. The phrase “effective amount” as used herein refers to an amount of a compound, material, or composition which is effective for producing some desired effect in at least a sub-population of cells. For example, a population of neural cells is contacted with an amount of an osteocrin-inducing agent described herein sufficient to produce a statistically significant, measurable response, e.g., a significant increase in neuronal connectivity, neuronal survival and/or axonal growth in the population of neural cells, when compared to a control population of neural cells. As used herein, the phrase “control population of neural cells” can refer to the same population of neural cells prior to treatment with an osteocrin-inducing agent or a separate population of neural cells without treatment with an osteocrin-inducing agent. In some embodiments, the “control population of neural cells” can refer to the same population of neural cells being treated at a prior time point. For in vivo applications, the “control population of neural cells” can be present in a normal healthy subject, or in a target subject to be treated (prior to the treatment with an osteocrin-inducing agent), or in a target subject already treated at a prior time point.

In some embodiments, a population of neural cells can be contacted with an effective amount of an osteocrin-inducing agent sufficient to increase the neuronal connectivity of the population of neural cells in vitro, ex vivo or in vivo (e.g., in a cell culture or in a human subject) by at least about 10%, including, e.g., at least about 20%, at least about 30%, at least about 40%, at least about 50%, at least about 60%, at least about 70%, at least about 80%, at least about 90%, at least about 95%, at least about 98% or higher, as compared to a control population of neural cells described herein. In some embodiments, at least one osteocrin-inducing agent can be provided to the neural cells in an effective amount sufficient to increase the neuronal connectivity of the population of neural cells in vitro, ex vivo or in vivo (e.g., in a cell culture or in a human subject) by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or higher, as compared to a control population of neural cells described herein. The neuronal connectivity of the population of neural cells can be measured and/or determined in vitro, ex vivo, or in vivo by any methods known in the art. For example, in some embodiments of this aspect and other aspects described herein, the neuronal connectivity can be determined and/or monitored with an imaging system, e.g., a microscope, and/or a functional magnetic resonance imaging, alone or in combination with use of at least one or more neural markers.

As described earlier, in some embodiments, an increase in neuronal connectivity can be characterized and/or measured by one or more neuronal connectivity indicators, e.g., but not limited to, an increase in dendritic density in the population of neural cells, an increase in the total dendritic length of the neural cells, an increase in the excitatory synapse density in the population of neural cells, an increase in neural cell survival, an increase in axonal growth of neural cells, an increase in cognitive function of a subject (for in vivo applications) or any combinations thereof. Methods for measuring one or more of these neuronal connectivity indicators are known in the art and have also been described above.

Accordingly, in some embodiments, a population of neural cells can be contacted with an effective amount of an osteocrin-inducing agent sufficient to increase the dendritic density of the population of neural cells in vitro, ex vivo or in vivo (e.g., in a cell culture or in a human subject) by at least about 10%, including, e.g., at least about 20%, at least about 30%, at least about 40%, at least about 50%, at least about 60%, at least about 70%, at least about 80%, at least about 90%, at least about 95%, at least about 98% or higher, as compared to a control population of neural cells described herein. In some embodiments, at least one or more osteocrin-inducing agents can be provided to the neural cells in an effective amount sufficient to increase the dendritic density of the population of neural cells in vitro, ex vivo or in vivo (e.g., in a cell culture or in a human subject) by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or higher, as compared to a control population of neural cells described herein.

In other embodiments, a population of neural cells (e.g., in a cell culture or in a human subject) can be contacted in vitro, ex vivo or in vivo with an effective amount of an osteocrin-inducing agent sufficient to increase the total dendritic length of the population of neural cells (e.g., in a cell culture or in a human subject) by at least about 10%, including, e.g., at least about 20%, at least about 30%, at least about 40%, at least about 50%, at least about 60%, at least about 70%, at least about 80%, at least about 90%, at least about 95%, at least about 98% or higher, as compared to a control population of neural cells described herein. In some embodiments, at least one or more osteocrin-inducing agent can be provided to the neural cells in vitro, ex vivo or in vivo in an effective amount sufficient to increase the total dendritic length of the population of neural cells (e.g., in a cell culture or in a human subject) by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or higher, as compared to a control population of neural cells described herein.

In some embodiments, a population of neural cells (e.g., in a cell culture or in a human subject) can be contacted in vitro, ex vivo or in vivo with an effective amount of an osteocrin-inducing agent sufficient to increase the excitatory synapse density of the population of neural cells (e.g., in a cell culture or in a human subject) by at least about 10%, including, e.g., at least about 20%, at least about 30%, at least about 40%, at least about 50%, at least about 60%, at least about 70%, at least about 80%, at least about 90%, at least about 95%, at least about 98% or higher, as compared to a control population of neural cells described herein. In some embodiments, the osteocrin-inducing agent can be provided to the neural cells in vitro, ex vivo or in vivo in an effective amount sufficient to increase the excitatory synapse density of the population of neural cells (e.g., in a cell culture or in a human subject) by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or higher, as compared to a control population of neural cells described herein.

In some embodiments, a population of neural cells (e.g., in a cell culture or in a human subject) can be contacted in vitro, ex vivo or in vivo with an effective amount of an osteocrin-inducing agent sufficient to increase the neuronal survival of the population of neural cells (e.g., in a cell culture or in a human subject) by at least about 10%, including, e.g., at least about 20%, at least about 30%, at least about 40%, at least about 50%, at least about 60%, at least about 70%, at least about 80%, at least about 90%, at least about 95%, at least about 98% or higher, as compared to a control population of neural cells described herein. In some embodiments, at least one or more osteocrin-inducing agent can be provided to the neural cells in vitro, ex vivo or in vivo in an effective amount sufficient to increase the neuronal survival of the population of neural cells (e.g., in a cell culture or in a human subject) by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or higher, as compared to a control population of neural cells described herein. In one embodiment, an osteocrin-inducing agent can be delivered to neural cells (e.g., in vitro, ex vivo or in vivo) or administered to a subject in an effective amount sufficient to increase the neuronal survival of the population of neural cells by at least about 2-fold or higher.

In some embodiments, a population of neural cells (e.g., in a cell culture or in a human subject) can be contacted in vitro, ex vivo or in vivo with an effective amount of an osteocrin-inducing agent sufficient to regenerate at least one or more axons, or increase axonal growth of a population of neural cells (e.g., in a cell culture or in a human subject) by at least about 10%, including, e.g., at least about 20%, at least about 30%, at least about 40%, at least about 50%, at least about 60%, at least about 70%, at least about 80%, at least about 90%, at least about 95%, at least about 98% or higher, as compared to a control population of neural cells described herein. In some embodiments, at least one or more osteocrin-inducing agent can be provided to the neural cells in vitro, ex vivo or in vivo in an effective amount sufficient to regenerate at least one or more axons, or increase axonal growth of a population of neural cells (e.g., in a cell culture or in a human subject) by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or higher, as compared to a control population of neural cells described herein. In one embodiment, an osteocrin-inducing agent can be delivered to neural cells (e.g., in vitro, ex vivo or in vivo) or administered to a subject in an effective amount sufficient to regenerate at least one or more axons, or increase axonal growth of a population of neural cells by at least about 2-fold or higher, including, e.g., at least about 3-fold, at least about 4-fold or higher.

In some embodiments where an osteocrin-inducing agent is administered to a subject in need of neuronal connectivity, neuronal survival, and/or axonal growth and/or regeneration, e.g., in his/her brain, an osteocrin-inducing agent can be administered to the subject in an amount sufficient to increase at least one or more cognitive functions (e.g., but not limited to, mental stability, memory/recall abilities, problem solving abilities, reasoning abilities, thinking abilities, judging abilities, capacity for learning, perception, intuition, awareness or any combinations thereof) of the subject by at least about 10%, including, e.g., at least about 20%, at least about 30%, at least about 40%, at least about 50%, at least about 60%, at least about 70%, at least about 80%, at least about 90%, at least about 95%, at least about 98% or higher, as compared to a control subject (e.g., the same subject prior to the administration of the osteocrin-inducing agent, or the same subject previously treated at a prior time point, or a control subject without the administration of the osteocrin-inducing agent). In some embodiments, the osteocrin-inducing agent can be provided to the neural cells in an effective amount sufficient to increase at least one or more cognitive functions (e.g., but not limited to, mental stability, memory/recall abilities, problem solving abilities, reasoning abilities, thinking abilities, judging abilities, capacity for learning, perception, intuition, awareness or any combinations thereof) of the subject by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or higher, as compared to a control subject (e.g., the same subject prior to the administration of the osteocrin-inducing agent, or the same subject previously treated at a prior time point, or a control subject without the administration of the osteocrin-inducing agent).

In some embodiments where the neural cells to be treated are motor neurons or sensory neurons, one or more osteocrin-inducing agents can be administered to a subject in an amount sufficient to increase at least one or more motor and/or sensory functions (e.g., but not limited to, perception, hearing, tactile senses, locomotion, motor coordination and/or endurance, and/or vestibular function) associated with the motor neurons or sensory neurons in need thereof, e.g., by at least about 10%, including, e.g., at least about 20%, at least about 30%, at least about 40%, at least about 50%, at least about 60%, at least about 70%, at least about 80%, at least about 90%, at least about 95%, at least about 98% or higher, as compared to a control subject (e.g., the same subject prior to the administration of the osteocrin-inducing agent, or the same subject previously treated at a prior time point, or a control subject without the administration of the osteocrin-inducing agent). In some embodiments, the osteocrin-inducing agent can be provided to the neural cells in an effective amount sufficient to increase at least one motor and/or sensory functions of the subject by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold or higher, as compared to a control subject (e.g., the same subject prior to the administration of the osteocrin-inducing agent, or the same subject previously treated at a prior time point, or a control subject without the administration of the osteocrin-inducing agent).

In some embodiments, the osteocrin-inducing agent can be introduced to a population of neural cells (e.g., human neural cells) in vitro, ex vivo or in vivo at an effective amount of about 0.1 ng/mL to about 100 μg/mL, or from about 0.5 ng/mL to about 50 ng/mL.

For in vivo applications, the osteocrin-inducing agent can be administered to a subject (e.g., a human subject) such that the extracellular or interstitial concentration of osteocrin ranges from about 0.1 ng/mL to about 100 μg/mL, or from about 0.5 ng/mL to about 50 ng/mL. In some embodiments, the osteocrin-inducing agent can be administered to a subject (e.g., a human subject) at an effective amount of about 0.1 ng/kg to about 20 mg/kg of body weight of the subject, or about 0.5 ng/kg to about 10 mg/kg of body weight of the subject, or about 1 ng/kg to about 5 mg/kg of body weight of the subject, or about 10 ng/kg to about 1 mg/kg of body weight of the subject. In some embodiments, the osteocrin-inducing agent can be administered to a subject at an effective amount of about 0.1 ng/kg to about 500 mg/kg body mass of the subject. In some embodiments, the osteocrin-inducing agent can be administered to a subject at an effective amount of about 1 mg/kg to about 250 mg/kg body mass of the subject. In some embodiments, the osteocrin-inducing agent can be administered to a subject at an effective amount of about 50 mg/kg to about 150 mg/kg body mass of the subject.

The effective amount can be administered to a subject in one dose or in separate doses, which can be taken at the same time or at different times. The effective amount of an osteocrin-inducing agent can vary with each individual subject or population of neural cells, depending on, e.g., but not limited to, condition of neural cells (e.g., degree of neurodegeneration), physical and/or clinical condition of each individual subject, potency and/or half-life of the osteocrin-inducing agent, administration frequency and/or routes, and any combinations thereof.

### Compositions Comprising an Osteocrin-Inducing Agent

Compositions comprising an effective amount of an osteocrin-inducing agent described herein for use in increasing neuronal connectivity, neuronal survival and/or axonal growth of a population of neural cells are also provided herein. These compositions can be employed in the methods and/or assays of various aspects described herein. In some embodiments, the compositions described herein can be employed in in vitro applications. In some embodiments, the compositions described herein can be employed in ex vivo applications such as treatment of neurodegenerative disease or disorder; or promotion of axonal growth and/or regeneration. In some embodiments, the compositions described herein can be employed in in vivo applications such as treatment, diagnosis and/or monitoring of a neurodegenerative condition and/or disorder in a subject, e.g., a human subject, and/or assessing efficacy of a treatment for a neurodegenerative condition in a subject.

In some embodiments, the compositions are formulated for cell-culture applications. For example, the cell-culture composition, in some embodiments, can further comprise a cell culture medium. As used herein, the term “cell culture medium” refers to any nutrient medium in which neural cells can be cultured in vitro. Examples of nutrients essential to cell metabolism and proliferation, e.g., amino acids, lipids, carbohydrates, vitamins and mineral salts can be included in the cell culture medium. In one embodiment, cell culture medium can also comprise any substance essential to neural cell differentiation. One of skill in the art can determine an appropriate formulation of cell culture medium for culturing neural cells, based on the cell condition (e.g., morphology, viability, growth rate, and/or cell density).

In some embodiments, the compositions can be formulated for pharmaceutical usages, e.g., therapeutic treatment. In these embodiments, the compositions can be formulated for neuro-protective purposes.

In some embodiments of this aspect and other aspects described herein, the composition or pharmaceutical composition can further comprise one or more neural stem cells. In some embodiments, the neural stem cells can be mammalian stem cells such as human stem cells. Examples of stem cells can include, but are not limited to, mesenchymal stem cells, embryonic stem cells, induced pluripotent stem cells, induced stem cells, cord blood-derived multipotent stem cells, bone marrow-derived stem cells, and any combinations thereof.

In some embodiments, the neural stem cells can be engineered to express and/or secrete osteocrin or an analog thereof at a level higher than (e.g., at least about 5% or higher, at least about 10% or higher than) that of control neural cells, e.g., target neural cells to be treated. In some embodiment, the neural cells transduced with a vector encoding an osteocrin-inducing agent can be included in the compositions and stored frozen. In such embodiments, an additive or preservative known for freezing cells can be included in the compositions. A suitable concentration of the preservative can vary from 0.02% to 2% based on the total weight although there may be appreciable variation depending upon the preservative or additive selected. One example of such additive or preservative can be dimethyl sulfoxide (DMSO) or any other cell-freezing agent known to a skilled artisan. In such embodiments, the composition can be thawed before use or administration to a subject, e.g., neuronal stem cell therapy.

In some embodiments of this aspect and other aspects described herein, the composition or pharmaceutical composition can further comprise at least one or more therapeutic agents (e.g., at least 1, at least 2, at least 3, at least 4 or more). As used herein, the term “therapeutic agent” generally means a molecule, group of molecules, complex or substance administered to an organism for diagnostic, therapeutic, preventative medical, or veterinary purposes. As used herein, the term “therapeutic agent” includes a “drug.” This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA), ribonucleic acid (RNA), or mixtures or combinations thereof, including, for example, DNA nanoplexes.

The term “therapeutic agent” also includes an agent that is capable of providing a local or systemic biological, physiological, or therapeutic effect in the biological system to which it is applied. For example, the therapeutic agent can act to control or inhibit infection or inflammation, enhance axonal growth and/or regeneration, act as an analgesic, promote anti-cell attachment, and enhance nerve growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. In some embodiments, different types of therapeutic agents that can be administered in combination with (e.g., prior to, in concurrent with, or after administration of) at least one osteocrin-inducing agent can include, but not limited to, proteins, peptides, antigens, immunogens, vaccines, antibodies or portions thereof, antibody-like molecules, enzymes, nucleic acids, siRNA, shRNA, aptamers, small molecules, antibiotics, antimicrobials, wound healing agents, and any combinations thereof. Exemplary therapeutic agents include, but are not limited to, those found in Harrison's Principles of Internal Medicine, 13th Edition, Eds. T. R. Harrison et al. McGraw-Hill N. Y., NY; Physicians Desk Reference, 50th Edition, 1997, Oradell N.J., Medical Economics Co.; Pharmacological Basis of Therapeutics, 8th Edition, Goodman and Gilman, 1990; United States Pharmacopeia, The National Formulary, USP XII NF XVII, 1990, the complete contents of all of which are incorporated herein by reference. In some embodiments, examples of therapeutic agents can include, but are not limited to, anti-inflammatory agents, anti-infective agents (including antibacterial, antifungal, antiviral, antiprotozoal agents), anti-allergic agents, anti-proliferative agents, anti-angiogenic agents, anti-oxidants, neuroprotective agents, cell receptor agonists, cell receptor antagonists, immunomodulating agents, immunosuppressive agents, intraocular pressure(IOP)-lowering agents (anti-glaucoma), beta adrenoceptor antagonists, alpha-2 adrenoceptor agonists, carbonic anhydrase inhibitors, cholinergic agonists, prostaglandins and prostaglandin receptor agonists, AMPA receptor antagonists, NMDA antagonists, angiotensin receptor antagonists, somatostatin agonists, mast cell degranulation inhibitors, alpha-2 adrenoceptor antagonists, thromboxane A2 mimetics, protein kinase inhibitors, prostaglandin F derivatives, prostaglandin-2 alpha antagonists, muscarinic agents, neuroprotective agents, and any combinations thereof.

In some embodiments, at least one therapeutic agent to be administered in combination with (e.g., prior to, in concurrent with, or after administration of) at least one osteocrin-inducing agent can include a therapeutic agent for treatment of a neurodegenerative and/or neurological condition and/or disorder described herein, a neuroprotective agent, a therapeutic agent to reduce infection and/or inflammation, a therapeutic agent to facilitate wound healing, and any combinations thereof.

In some embodiments, as described earlier, an expression vector can be used to express and deliver at least one or more osteocrin-inducing agents (e.g., an osteocrin-inducing nucleic acid molecule) into a population of neural cells in situ. In such embodiments, the composition can comprise a concentration of viral vectors from about 10⁴ viral genomes/ml to about 10²⁰ viral genomes/ml, from about 10⁵ viral genomes/ml to about 10¹⁸ viral genomes/ml, from about 10⁶ viral genomes/ml to about 10¹⁵ viral genomes/ml, or from about 10¹⁰ viral genomes/ml to about 10¹⁵ viral genomes/ml. In some embodiments, the composition can comprise a concentration of viral vectors from about 1×10¹² viral genomes/ml to about 1×10¹³ viral genomes/ml. Depending on the use of compositions described herein, a skilled artisan can determine an appropriate concentration of the viral vectors in a composition. For example, for cell culture compositions, e.g., comprising a cell culture medium, lower concentrations of viral vectors, e.g., 1×10⁵ viral genomes/ml-1×10⁸ viral genomes/ml can be selected for a culturing purpose. For therapeutic administration purpose, the composition can comprise higher concentrations of viral vectors, e.g., about 1×10¹⁰ viral genomes/ml to about 1×10¹⁵ viral genomes/ml. The precise determination of an effective dose can be based on individual factors, including their physical and/or clinical condition (e.g., age, and/or amount of time since neurodegeneration). Therefore, dosages can be readily adjusted for each individual patient by those skilled in the art.

For administration to a subject in need thereof, e.g., a subject diagnosed with having, or having a risk for, a neurodegenerative condition, e.g., but not limited to, Alzheimer's disease, the osteocrin-inducing agent and/or viral expression vector encoding the osteocrin-inducing agent can be provided in a pharmaceutically acceptable composition. As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The pharmaceutically acceptable composition can further comprise one or more pharmaceutically carriers (additives) and/or diluents. As used herein, the term “pharmaceutically-acceptable carrier” refers to a carrier that is physiologically acceptable to the treated subject while retaining the therapeutic properties of the compound with which it is administered. Some examples of materials which can serve as pharmaceutically-acceptable carriers include, but are not limited to, physiological saline, artificial interstitial fluid, gelatin, buffering agents, such as magnesium hydroxide and aluminum hydroxide, pyrogen-free water, isotonic saline, Ringer's solution, pH buffered solutions, bulking agents such as polypeptides and amino acids, serum component such as serum albumin, HDL and LDL, and other non-toxic compatible substances employed in pharmaceutical formulations. Preservatives and antioxidants can also be present in the formulation. Other physiologically acceptable carriers and their formulations are known to one skilled in the art and described, for example, in Remington's Pharmaceutical Sciences, (20th edition), ed. A. Gennaro, 2000, Lippincott, Williams & Wilkins, Philadelphia, Pa.

Pharmaceutically acceptable carriers can vary in compositions comprising an osteocrin-inducing agent described herein, depending on the administration route and/or formulation. For example, the pharmaceutically acceptable composition can be delivered via injection. These routes for administration (delivery) include, but are not limited to, subcutaneous or parenteral including intravenous, intracortical, intracranial, intramuscular, intraperitoneal, and infusion techniques. In one embodiment, the pharmaceutical acceptable composition can be in a form that is suitable for intracortical injection. In another embodiment, the pharmaceutical composition is formulated for intracranial injection. Other forms of administration can be also employed, e.g., oral, systemic, inhalation or parenteral administration.

The osteocrin-inducing agent and/or the composition comprising the same can be formulated in pharmaceutically acceptable compositions, together with one or more pharmaceutically acceptable carriers (additives) and/or diluents. The compounds can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally, for example, a spray or an aerosol. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. Nos. 3,773,919; 3,270,960; 3,845,770; 3,916,899; 3,536,809; 3,598,123; and 4,008,719.

When administering a pharmaceutical composition parenterally, it will be generally formulated in a unit dosage injectable form (solution, suspension, emulsion). The pharmaceutical formulations suitable for injection include sterile aqueous solutions or dispersions. The carrier can be a solvent or dispersing medium containing, for example, water, cell culture medium, buffers (e.g., phosphate buffered saline), polyol (for example, glycerol, propylene glycol, liquid polyethylene glycol, and the like), and suitable mixtures thereof. In some embodiments, the pharmaceutical carrier can include a physiological saline.

In some embodiments, the pharmaceutical composition can be formulated in an emulsion or a gel. In such embodiments, at least one osteocrin-inducing agent or viral vector encoding an osteocrin-inducing agent can be encapsulated within a biocompatible gel, e.g., hydrogel and a peptide gel. The gel pharmaceutical composition can be implanted to the brain near the degenerating neural cells.

In some embodiments, the composition can be formulated for sustained delivery. As used herein, the term “sustained delivery” is refers to continual delivery of an osteocrin-inducing agent (and optionally a therapeutic agent) in vivo or in vitro over a period of time following administration. For example, sustained release can occur over a period of at least several days, a week, several weeks, and a year or longer. Sustained delivery of the osteocrin-inducing agent (and optionally a therapeutic agent) in vivo can be demonstrated by, for example, the continued therapeutic effect of the osteocrin-inducing agent (and optionally the therapeutic agent) over time. Alternatively, sustained delivery of the agent may be demonstrated by detecting the presence of the osteocrin-inducing agent (and optionally a therapeutic agent) in vivo over time. In some embodiments, the sustain release can be over a period of one week, two weeks, three weeks, four weeks, one month, two months, three months, four months, five months, six months or longer.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like.

The compositions can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON'S PHARMACEUTICAL SCIENCE”, 20th edition, 2000, incorporated herein by reference, may be consulted to prepare suitable preparations, without undue experimentation. With respect to compositions described herein, however, any vehicle, diluent, or additive used should be biocompatible or inert with the osteocrin-inducing agent or a vector encoding the osteocrin-inducing agent.

The compositions can be isotonic, i.e., they can have the same osmotic pressure as in the fluid or in the tissue to where the compositions are administered. The desired isotonicity of the compositions described herein can be accomplished using sodium chloride, or other pharmaceutically acceptable agents such as dextrose, boric acid, sodium tartrate, propylene glycol or other inorganic or organic solutes. In one embodiment, sodium chloride is used in buffers containing sodium ions.

Viscosity of the compositions can be maintained at the selected level using a pharmaceutically acceptable thickening agent. In one embodiment, methylcellulose is used because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

Typically, any additives (in addition to the osteocrin-inducing agent) can be present in an amount of 0.001 to 50 wt % solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, and about 0.05 to about 5 wt %. For any therapeutic composition to be administered to a subject in need thereof, and for any particular method of administration, it is preferred to determine toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan.

The compositions described herein can be prepared by mixing the ingredients following generally-accepted procedures. For example, an effective amount of an osteocrin-inducing agent or vectors encoding an osteocrin-inducing agent can be re-suspended in an appropriate pharmaceutically acceptable carrier and the mixture can be adjusted to the final concentration and viscosity by the addition of water or thickening agent and possibly a buffer to control pH or an additional solute to control tonicity. An effective amount of at least one osteocrin-inducing agent described herein and any other additional agent, e.g., for inhibiting neurodegeneration, can be mixed with the cell mixture. Generally the pH can vary from about 3 to about 7.5. In some embodiments, the pH of the composition can be about 6.5 to about 7.5. Compositions can be administered in dosages and by techniques well known to those skilled in the medical and veterinary arts taking into consideration such factors as the age, sex, weight, and condition of the particular patient, and the composition form used for administration (e.g., liquid). Dosages for humans or other mammals can be determined without undue experimentation by a skilled artisan.

### Selection of a Subject for Methods of Treatment Described Herein

The methods of treatment described herein can be used to treat a subject, who is determined to have, or have a risk, for cognitive impairment, motor neuron impairment and/or sensory neuron impairment. The methods of treatment described herein can also be used to treat a subject, who is determined to have, or have a risk for having a neurodegenerative condition, e.g., but not limited to, Alzheimer's disease. In some embodiments, the method of treatment can further comprise selecting a subject (e.g., a human subject) who is determined to have, or have a risk for having, cognitive impairment, motor function impairment (e.g., but not limited to, impairment in locomotion, upper/lower extremity movements) and/or sensory function impairment (e.g., but not limited to, perception, hearing, and/or tactile senses), prior to administration with a composition comprising an osteocrin-inducing agent. In some embodiments, the method of treatment can further comprise selecting a subject (e.g., a human subject) who is determined to have, or have a risk for having, a neurodegenerative condition, e.g., but not limited to, Alzheimer's disease, prior to administration with a composition comprising an osteocrin-inducing agent.

The phrase “having a risk for,” when used in reference to a disease, disorder or condition, refers to a subject presenting one or more symptoms indicative of the disease, disorder or condition. By way of example only, a subject having a risk for cognitive impairment can present at least one or more symptoms associated with a cognitive function, e.g., but not limited to, problems with memory, orientation, language, information processing, the ability to focus and sustain attention on a task, decrease in motor skills, etc. As used herein, the term “cognitive function” can encompass motor and/or sensory functions.

Methods for evaluating cognitive function and/or diagnosing for a neurodegenerative condition, e.g., but not limited to, Alzheimer's disease, are known in the art, e.g., using various types of neurophysiological tests such as assessment tests for intelligence, memory, language, executive function, and/or visuospatial; dementia-specific tests, and/or batteries assessing multiple neuropsychological functions; as well as imaging methods, such as functional magnetic imaging resonance, PET; biological and/or genetic testings.

By way of example only, methods for diagnosing a specific neurodegenerative condition such as Alzheimer's disease are well known in the art. For example, the stage of Alzheimer's disease can be assessed using the Functional Assessment Staging (FAST) scale, which divides the progression of Alzheimer's disease into 16 successive stages under 7 major headings of functional abilities and losses: Stage 1 is defined as a normal adult with no decline in function or memory. Stage 2 is defined as a normal older adult who has some personal awareness of functional decline, typically complaining of memory deficit and forgetting the names of familiar people and places. Stage 3 (early Alzheimer's disease) manifests symptoms in demanding job situation, and is characterized by disorientation when traveling to an unfamiliar location; reports by colleagues of decreased performance; name- and word-finding deficits; reduced ability to recall information from a passage in a book or to remember a name of a person newly introduced to them; misplacing of valuable objects; decreased concentration. In stage 4 (mild Alzheimer's Disease), the patient may require assistance in complicated tasks such as planning a party or handling finances, exhibits problems remembering life events, and has difficulty concentrating and traveling. In stage 5 (moderate Alzheimer's disease), the patient requires assistance to perform everyday tasks such as choosing proper attire. Disorientation in time, and inability to recall important information of their current lives, occur, but patient can still remember major information about themselves, their family and others. In stage 6 (moderately severe Alzheimer's disease), the patient begins to forget significant amounts of information about themselves and their surroundings and require assistance dressing, bathing, and toileting. Urinary incontinence and disturbed patterns of sleep occur. Personality and emotional changes become quite apparent, and cognitive abulia is observed. In stage 7 (severe Alzheimer's disease), speech ability becomes limited to just a few words and intelligible vocabulary may be limited to a single word. A patient can lose the ability to walk, sit up, or smile, and eventually cannot hold up the head.

Other alternative diagnostic methods for AD include, but not limited to, cellular and molecular testing methods disclosed in U.S. Pat. No. 7,771,937, U.S. Pat. No. 7,595,167, U.S. Pat. No. 5,558,0748, and PCT Application No.: WO2009/009457, the content of which is incorporated by reference. Additionally, protein-based biomarkers for AD, some of which can be detected by non-invasive imaging, e.g., PET, are disclosed in U.S. Pat. No. 7,794,948, the content of which is incorporated by reference.

Genes involved in AD risk can be used for diagnosis of AD. One example of other AD risk genes is apolipoprotein E-ε4 (APOE-ε4). APOE-ε4 is one of three common forms, or alleles, of the APOE gene; the others are APOE-e2 and APOE-e3. APOE provides the blueprint for one of the proteins that carries cholesterol in the bloodstream. Everyone inherits a copy of some form of APOE from each parent. Those who inherit one copy of APOE-ε4 have an increased risk of developing AD. Those who inherit two copies have an even higher risk, but not a certainty of developing AD. In addition to raising risk, APOE-ε4 may tend to make symptoms appear at a younger age than usual. Other AD risk genes in addition to APOE-e4 are well established in the art. Some of them are disclosed in US Pat. App. No.: US 2010/0249107, US 2008/0318220, US 2003/0170678 and PCT Application No.: WO 2010/048497, the content of which is incorporated by reference. Genetic tests are well established in the art and are available, for example for APOE-e4. A subject carrying the APOE-ε4 allele can, therefore, be identified as a subject at risk of developing AD.

In further embodiments, subjects with Aβ burden are amenable to the methods of treatment described herein. Such subjects include, but not limited to, the ones with Down syndrome, Huntington disease, the unaffected carriers of APP or presenilin gene mutations, and the late onset AD risk factor, apolipoprotein E-ε4.

In some embodiments, subjects who are currently receiving a medication or a treatment regimen associated with a neurodegenerative condition, e.g., but not limited to, AD, can also be subjected to the methods of treatment as described herein.

In some embodiments, a subject who has been diagnosed with an increased risk for developing a neurodegenerative condition, e.g., using the diagnostic methods and assays described below, can be subjected to the methods of treatment as described herein.

As used herein, a “subject” can mean a human or an animal. Examples of subjects include primates (e.g., humans, and monkeys). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. A patient or a subject includes any subset of the foregoing, e.g., all of the above, or includes one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

In one embodiment, the subject is a mammal. The mammal can be a human, non-human primate, mouse, rat, dog, cat, horse, or cow, but are not limited to these examples. Mammals other than humans can be advantageously used as subjects that represent animal models of neural stem cell therapy. In addition, the methods and compositions described herein can be employed in domesticated animals and/or pets. In one embodiment, the subject amenable to the methods described herein is a human subject.

### Use of Osteocrin as a Diagnostic Marker

Without wishing to be bound by theory, as osteocrin is a neural activity-regulated gene, particularly in a human subject, in some embodiments, osteocrin can be used as a neural biomarker to determine if a subject has, or has a risk for developing a neurodegenerative condition and/or disorder. Accordingly, a further aspect described herein relates to an assay for determining a subject's susceptibility to, or risk for, developing a neurodegenerative condition and/or disorder. The assay can comprise (a) subjecting a test sample of a subject, who is determined to have, or have displayed symptoms of cognitive impairment, to at least one analysis to determine expression of osteocrin; and (b) comparing the expression of osteocrin with a reference value using a non-human machine, e.g., a specifically-programmed computer. In some embodiments, the assay can further comprise administering to the subject an osteocrin-inducing agent if the comparison indicates that a subject is diagnosed as having, or having a risk for, a neurodegenerative condition and/or disorder.

In some embodiments, the reference value can correspond to expression of osteocrin in a normal healthy subject. In such embodiments, a lower expression of osteocrin determined in the test sample of the subject than that of the normal healthy subject, e.g., a reduction of at least about 10%, including, e.g., a reduction of at least about 20%, at least about 30%, at least about 40%, at least about 50%, at least about 60%, at least about 70% or more, can be indicative of the subject diagnosed as having, or having a risk for a neurodegenerative condition and/or disorder.

In some embodiments, the reference value can correspond to expression of osteocrin in a control subject diagnosed with a neurodegenerative condition and/or disorder. In such embodiments, a higher expression of osteocrin determined in the test sample of the subject that that of the control subject, e.g., by no more than 50%, can be indicative of the subject diagnosed as having, or having a risk for, a neurodegenerative condition and/or disorder. In some embodiments, a higher expression of osteocrin determined in the test sample of the subject than that of the control subject, e.g., by no more than 40%, no more than 30%, no more than 20%, no more than 10%, no more than 5% or less, can be indicative of the subject diagnosed as having, or having a risk for, a neurodegenerative condition and/or disorder.

In some embodiments, the reference value can correspond to osteocrin expression of a test sample collected from a target subject at a prior time point. In such embodiments, a reduced level of osteocrin expression in a test sample collected from the target subject collected at a later time point, e.g., a reduction of at least about 10%, including, e.g., a reduction of at least about 20%, at least about 30%, at least about 40%, at least about 50%, at least about 60%, at least about 70% or more, can be indicative of the subject diagnosed as having, or having a risk for, a neurodegenerative condition and/or disorder.

In some embodiments, the test sample to be analyzed can comprise a blood sample collected from a subject (e.g., a human subject). In some embodiments, the test sample to be analyzed can comprise a brain biopsy obtained from a subject (e.g., a human subject).

Expression of osteocrin in a test sample of a subject can be determined by any analyses known in the art, depending on, e.g., types of test samples and/or abundance of osteocrin in test samples. For example, without limitations, western blot, enzyme linked absorbance assay, mass spectrometry, immunoassay, flow cytometry, immunohistochemical analysis, PCR reaction, real-time quantitative PCR, and any combinations thereof, can be used to determine expression (e.g., protein-level and/or transcript-level) of osteocrin in test samples.

The assay described herein can be used to diagnose a subject (e.g., a human subject) for any neurodegenerative condition described herein. In one embodiment, the assay described herein can be used to diagnose Alzheimer's disease.

Embodiments of various aspects described herein can be defined in any of the following numbered paragraphs:
1. A method of increasing neuronal connectivity of a population of neural cells comprising contacting the population of neural cells with a composition comprising an effective amount of an osteocrin-inducing agent.
2. The method of paragraph 1, wherein the increase in neuronal connectivity of the population of neural cells includes an increase in neuronal survival.
3. The method of paragraph 1 or 2, wherein the increase in neuronal connectivity of the population of neural cells includes an increase in axonal growth of at least one of the neural cells.
4. The method of any of paragraphs 1-3, wherein the osteocrin-inducing agent comprises a recombinant osteocrin protein or a peptidomimetic thereof.
5. The method of any of paragraphs 1-4, wherein the osteocrin-inducing agent comprises a recombinant osteocrin-encoding gene.
6. The method of paragraph 5, wherein the recombinant osteocrin-encoding gene is operably linked to an expression vector (e.g., viral vector, plasmid vector).
7. The method of any of paragraphs 1-6, wherein the composition further comprises a neural stem cell.
8. The method of any of paragraphs 1-7, wherein the osteocrin-inducing agent comprises a small molecule that induces expression of secreted osteocrin.
9. The method of paragraph 8, wherein the small molecule is a ligand for a natriuretic peptide clearance receptor.
10. The method of paragraph 8 or 9, wherein the small molecule excludes a PPAR-gamma agonist (e.g., Troglitazone).
11. The method of any of paragraphs 1-10, wherein the population of neural cells is present in a cell culture.
12. The method of paragraph 11, wherein the population of neural cells in the cell culture comprise human neural cells.
13. The method of any of paragraphs 1-10, wherein the population of neural cells is present in a subject.
14. The method of paragraph 13, wherein the subject is diagnosed as having, or having a risk for, cognitive impairment, motor function impairment, and/or sensory function impairment.
15. The method of paragraph 13 or 14, wherein the subject is determined to be in need of axonal growth and/or regeneration.
16. The method of any of paragraphs 13-15, wherein the subject is diagnosed as having, or having a risk for, a neurodegenerative condition.
17. The method of paragraph 16, wherein the neurodegenerative condition is Alzheimer's disease.
18. The method of any of paragraphs 13-17, further comprising selecting a subject diagnosed as having, or having a risk for, cognitive impairment, motor function impairment and/or sensory function impairment, or a neurodegenerative condition, prior to the contacting.
19. The method of any of paragraphs 13-18, wherein the subject is a human subject.
20. The method of any of paragraphs 1-19, wherein the effective amount of the osteocrin-inducing agent is sufficient to increase the neuronal connectivity of the population of neural cells by at least about 10%, as compared to a control population of neural cells not contacted with the osteocrin-inducing agent.
21. A method of treating a human subject diagnosed as having, or having a risk for, a neurodegenerative condition comprising administering to the human subject a pharmaceutical composition comprising an effective amount of an osteocrin-inducing agent sufficient to increase neuronal connectivity of a population of neural cells in the human subject by at least about 10%, as compared to administration in the absence of the osteocrin-inducing agent.
22. The method of paragraph 21, wherein the neurodegenerative condition is Alzheimer's disease.
23. The method of any of paragraphs 1-22, wherein the neuronal connectivity is determined with an imaging system.
24. The method of any of paragraphs 13-23, wherein an increase in neuronal connectivity is measured by determining an increase in cognitive function of the subject.
25. The method of any of paragraphs 13-24, wherein the effective amount of the osteocrin-inducing agent is sufficient to increase at least one cognitive function of the subject by at least about 10%, as compared to a control subject not administered with the osteocrin-inducing agent.
26. The method of any of paragraphs 13-25, wherein the effective amount of the osteocrin-inducing agent is sufficient to increase at least one motor function of the subject by at least about 10%, as compared to a control subject not administered with the osteocrin-inducing agent.
27. The method of any of paragraphs 13-24, wherein the effective amount of the osteocrin-inducing agent is sufficient to increase at least one sensory function of the subject by at least about 10%, as compared to a control subject not administered with the osteocrin-inducing agent.
28. The method of any of paragraphs 1-27, wherein an increase in neuronal connectivity is measured by determining an increase in dendritic density in the population of neural cells.
29. The method of any of paragraphs 1-28, wherein the effective amount of the osteocrin-inducing agent is sufficient to increase the dendritic density in the population of neural cells by at least about 10%, as compared to a control population of neural cells not contacted with the osteocrin-inducing agent.
30. The method of paragraph 28 or 29, wherein said an increase in the dendritic density is measured by determining an increased expression of at least one dendritic marker.
31. The method of any of paragraphs 1-30, wherein an increase in neuronal connectivity is measured by determining an increase in excitatory synapse density in the population of neural cells.
32. The method of any of paragraphs 1-31, wherein the effective amount of the osteocrin-inducing agent is sufficient to increase the excitatory synapse density in the population of neural cells by at least about 10%, as compared to a control population of neural cells not contacted with the osteocrin-inducing agent.
33. The method of paragraph 31 or 32, wherein said an increase in the excitatory synapse density is measured by determining an increased expression of at least one synaptic marker.
34. The method of paragraph 30 or 33, wherein said at least one dendritic marker or synaptic marker includes MAP2, PSD95, synapsin, or any combinations thereof.
35. The method of any of paragraphs 1-34, wherein an increase in neuronal connectivity is measured by determining an increase in neuronal survival in the population of neural cells.
36. The method of paragraph 35, wherein the effective amount of the osteocrin-inducing agent is sufficient to increase the neuronal survival in the population of neural cells by at least about 10%, as compared to a control population of neural cells not contacted with the osteocrin-inducing agent.
37. The method of any of paragraphs 1-36, wherein an increase in neuronal connectivity is measured by determining an increase in axonal growth of at least one of the neural cells.
38. The method of paragraph 37, wherein the effective amount of the osteocrin-inducing agent is sufficient to increase the axonal growth of the neural cell by at least about 10%, as compared to a control population of neural cells not contacted with the osteocrin-inducing agent.
39. The method of any of paragraphs 1-38, wherein the effective amount of the osteocrin-inducing agent ranges from about 0.1 ng/mL to about 100 μg/mL, or from about 0.5 ng/mL to about 50 ng/mL.
40. An assay for determining a risk for a neurodegenerative disorder in a subject comprising:
   a. subjecting a test sample of the subject, who is determined to have, or have symptoms of, cognitive impairment, motor function impairment and/or sensory function impairment, to at least one analysis to determine expression of osteocrin;
   b. comparing the expression of osteocrin with a reference value using a non-human machine, and
   c. optionally administering to the subject an osteocrin-inducing agent if the comparison indicates that a subject is diagnosed as having, or having a risk for, a neurodegenerative disorder.
41. The assay of paragraph 40, wherein when the reference value corresponds to expression of osteocrin in a normal healthy subject, an expression of osteocrin determined in the test sample being lower than the reference value by at least about 10% is indicative of the subject diagnosed as having, or having a risk for, a neurodegenerative disorder.
42. The assay of paragraph 40, wherein when the reference value corresponds to expression of osteocrin in a control subject diagnosed with cognitive impairment or a neurodegenerative disorder, an expression of osteocrin determined in the test sample being higher than the reference value by no more than 50% is indicative of the subject diagnosed as having, or having a risk for, a neurodegenerative disorder.
43. The assay of any of paragraphs 40-42, wherein said at least one analysis is selected from the group consisting of western blot, enzyme linked absorbance assay, mass spectrometry, immunoassay, flow cytometry, immunohistochemical analysis, PCR reaction, real-time quantitative PCR, and any combinations thereof.
44. The assay of any of paragraphs 40-43, wherein the test sample comprises a blood sample.
45. The assay of any of paragraphs 40-44, wherein the neurodegenerative condition is Alzheimer's disease.
46. The assay of any of paragraphs 40-45, wherein the subject is a human subject.
47. A cell-culture or pharmaceutical composition comprising an effective amount of an osteocrin-inducing agent for use in increasing neuronal connectivity of a population of neural cells.
48. A neuro-protective composition comprising an effective amount of an osteocrin-inducing agent for use in the treatment or prevention of a neurodegenerative condition.
49. The composition of paragraph 48, wherein the neurodegenerative condition is Alzheimer's disease.
50. The composition of any of paragraphs 47-49, wherein the osteocrin-inducing agent comprises a recombinant human osteocrin protein or a peptidomimetic thereof.
51. The composition of any of paragraphs 47-50, wherein the osteocrin-inducing agent comprises a recombinant osteocrin-encoding gene.
52. The composition of paragraph 51, wherein the recombinant osteocrin-encoding gene is operably linked to an expression vector.
53. The composition of any of paragraphs 47-52, further comprising a neural stem cell.
54. The composition of any of paragraphs 47-53, wherein the osteocrin-inducing agent comprises a small molecule that induces expression of secreted osteocrin.
55. The composition of paragraph 54, wherein the small molecule is a ligand for a natriuretic peptide clearance receptor.
56. The composition of paragraph 54 or 55, wherein the small molecule excludes a PPAR-gamma agonist (e.g., Troglitazone).
57. The composition of any of paragraphs 47-56, wherein the neuronal connectivity is determined with an imaging system.
58. The composition of any of paragraphs 47-57, wherein an increase in neuronal connectivity is measured by determining an increase in cognitive function in a subject.
59. The composition of paragraph 58, wherein the effective amount of the osteocrin-inducing agent is sufficient to increase the cognitive function, motor function, and/or sensory function in the subject diagnosed with having, or having a risk, for the neurodegenerative condition, by at least about 10%, as compared to a control subject not administered with the osteocrin-inducing agent.
60. The composition of any of paragraphs 47-59, wherein an increase in neuronal connectivity is measured by determining an increase in dendritic density in the population of neural cells.
61. The composition of paragraph 60, wherein the effective amount of the osteocrin-inducing agent is sufficient to increase the dendritic density in the population of neural cells by at least about 10%, as compared to a control population of neural cells not contacted with the osteocrin-inducing agent.
62. The composition of paragraph 60 or 61, wherein the dendritic density is determined by expression of at least one dendritic marker in the population of neural cells, as compared to a control population of neural cells not contacted with the osteocrin-inducing agent.
63. The composition of any of paragraphs 47-62, wherein an increase in neuronal connectivity is measured by determining an increase in excitatory synapse density in the population of neural cells.
64. The composition of paragraph 63, wherein the effective amount of the osteocrin-inducing agent is sufficient to increase the excitatory synapse density in the population of neural cells by at least about 10%, as compared to a control population of neural cells not contacted with the osteocrin-inducing agent.
65. The composition of paragraph 63 or 64, wherein the excitatory synapse density is determined by expression of at least one synaptic marker.
66. The composition of paragraph 62 or 65, wherein said at least one dendritic marker or synaptic marker includes MAP2, PSD95, synapsin, or any combinations thereof.
67. The composition of any of paragraphs 47-66, wherein an increase in neuronal connectivity is measured by determining an increase in neuronal survival in the population of neural cells.
68. The composition of paragraph 67, wherein the effective amount of the osteocrin-inducing agent is sufficient to increase the neuronal survival in the population of neural cells by at least about 10%, as compared to a control population of neural cells not contacted with the osteocrin-inducing agent.
69. The composition of any of paragraphs 47-68, wherein an increase in neuronal connectivity is measured by determining an increase in axonal growth of at least one of the neural cells.
70. The composition of paragraph 69, wherein the effective amount of the osteocrin-inducing agent is sufficient to increase the axonal growth of the neural cell by at least about 10%, as compared to a control population of neural cells not contacted with the osteocrin-inducing agent.
71. The composition of any of paragraphs 47-70, wherein the effective amount of the osteocrin-inducing agent ranges from about 0.1 ng/mL to about 100 μg/mL, or from about 0.5 ng/mL to about 50 ng/mL.

### Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the paragraphed invention, because the scope of the invention is limited only by the paragraphs. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with numerical values may mean±5%.

The singular terms “a,” “an,” and “the” include plural referents unless context clearly indicates otherwise. Similarly, the word “or” is intended to include “and” unless the context clearly indicates otherwise. Thus for example, references to “the method” includes one or more methods, and/or steps of the type described herein and/or which will become apparent to those persons skilled in the art upon reading this disclosure and so forth.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The term “hydrogel” as used herein refers to natural or synthetic polymers that show superabsorbent properties (having even over 99% water) and possess a degree of flexibility similar to natural tissue, due to their significant water content. Examples of hydrogels used as scaffolds in tissue engineering or reservoirs in local drug delivery include, but are not limited to, methylcellulose, hyaluronan, and other naturally derived polymers. In one embodiment, the hydrogel is biodegradable.

The term “increase” as used herein generally means an increase by a statistically significant amount. In one embodiment, “increase” refers to an increase by at least 10% as compared to a reference level, for example an increase by at least about 20%, or at least about 30%, or at least about 40%, or at least about 50%, or at least about 60%, or at least about 70%, or at least about 80%, or at least about 90% or up to and including a 100% increase, or any increase between 10-100% as compared to a reference level. The reference level as used herein refers to a control in the absence of an osteocrin-inducing agent. In one embodiment, the reference level is measured in neural cells to be treated, prior to administration of the composition or osteocrin-inducing agent described herein.

The term “statistically significant” or “significantly” refers to statistical significance and generally means a two standard deviation (2 SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, a “prodrug” refers to compounds that can be converted via some chemical or physiological process (e.g., enzymatic processes and metabolic hydrolysis) to an active compound. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), content of all of which is herein incorporated by reference.

The term “analog” as used herein refers to a compound that results from substitution, replacement or deletion of various organic groups or hydrogen atoms from a parent compound. As such, some monoterpenoids can be considered to be analogs of monoterpenes, or in some cases, analogs of other monoterpenoids, including derivatives of monoterpenes. An analog is structurally similar to the parent compound, but can differ by even a single element of the same valence and group of the periodic table as the element it replaces.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

### Examples

The examples presented herein relate to identification of osteocrin as a human-specific neural activity-regulated gene that encodes a secreted protein capable of promoting neuronal activity, and enhancement and/or improvement of neuronal connectivity with an osteocrin-inducing agent, e.g., but not limited to a recombinant osteocrin protein. In some embodiments, the osteocrin-inducing agent can be administered to a population of neural cells in a subject who is determined to have, or have a risk for, a neurodegenerative condition, e.g., but not limited to Alzheimer's disease. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the paragraphs to the invention, but are rather intended to be exemplary of certain embodiments. Any variations in the exemplified methods which occur to the skilled artisan are intended to fall within the scope of the present invention.

### Example 1

### Identification of Osteocrin as a Human-Specific Neural Activity-Regulated Gene for Promotion of Human Neuronal Connectivity

Sensory, motor, and/or cognitive experiences generally trigger changes at neuronal synapses for proper brain development and/or higher cognitive function. These alterations can be mediated in part by the induction of new gene expression in neurons in response to elevated neural activity. In some model organisms, except in a human model, it has been shown that neuronal activity-regulated genes encode factors that mediate neuronal survival and can contribute to specific aspects of synaptic plasticity and/or neuronal connectivity. Moreover, inherited mutations in a number of the human homologues for these factors can give rise to a variety of human disorders of cognitive function. However, the nature of the neural activity-dependent gene program in primary human neurons had yet to be characterized.

To this end, the inventors have employed a primary human mixed cortical culture system in which the level of neuronal activity can be acutely modulated, together with next-generation DNA sequencing methods, to elucidate aspects of the neuronal activity-dependent gene response that are unique to human cultures. The inventors have identified osteocrin as a human-specific neural activity-regulated gene that encodes a secreted protein capable of promoting neuronal connectivity. Without wishing to be bound by theory, given data correlating loss of central nervous system neuronal connectivity with cognitive impairments in various neurodegenerative conditions, e.g., Alzheimer's disease, delivery of an osteocrin-inducing agent to neural cells can provide a therapeutic strategy to treat these devastating human disorders.

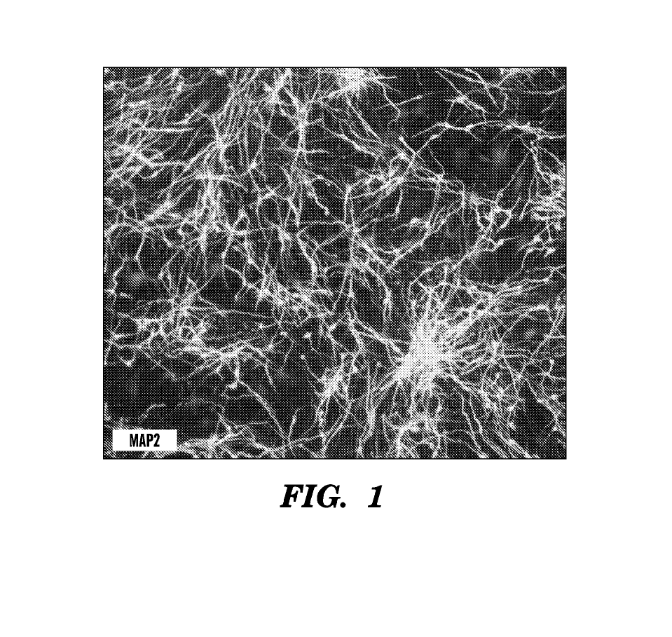

In this example, mixed cortical neuronal cultures, e.g., obtained from Sciencell Research Laboratories (Carlsbad, Calif.), were characterized and utilized to identify human-specific neural activity-regulated genes. These cells are derived from the discarded cerebral cortices of aborted midsecond trimester human fetuses. These cells, at least in part, adopt characteristic neuronal morphologies, express appropriate neuronal and/or synaptic markers, and/or assemble into synaptically connected neuronal networks that exhibit appropriate levels of basal activity (FIG. 1 and data not shown). Further, synchronous induction of neuronal activity, e.g., by means of potassium chloride (KCl)-triggered membrane depolarization, was found to robustly induce multiple known neural activity-responsive genes in these cultures.

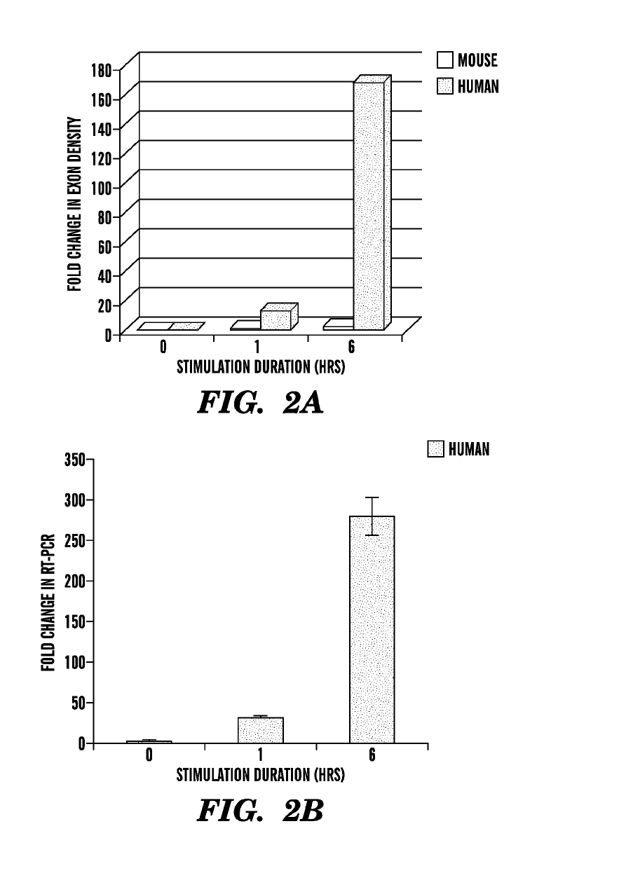

High-throughput RNA sequencing (RNA-seq) of cultures stimulated for 0, about 1 hour, or about 6 hours, e.g., with ˜55 mM KCl, was used to identify human RNA species that can be reproducibly unregulated in response to neuronal stimulation. Comparison of the identified human neural activity-regulated loci with those obtained from a parallel experimental system employing primary mouse cortical neurons isolated several human-specific neural activity-responsive genes, including, but not limited to, the gene encoding osteocrin (OSTN), a recently identified secreted protein produced by cells of the osteoblast lineage. In human cultures membrane depolarization triggered a dramatic increase in the level of OSTN mRNA, whereas OSTN mRNA levels in mouse cultures were unaffected by neuronal activity, remaining at the lower limits of experimental detection (FIG. 2A). Followup quantitative RT-PCR experiments indicated robust induction of the human OSTN mRNA in human mixed cortical cultures, and demonstrated that this induction is dependent on calcium influx through neuronal voltage-sensitive calcium channels (FIG. 2B). RT-PCR analysis of human brain RNA samples also indicated the presence of OSTN mRNA, consistent with data from the Human Brain Transcriptome database, which reports human OSTN mRNA levels increasing specifically in the neocortex during fetal brain development.

### Example 2

### Effects on an Osteocrin-Inducing Agent on Human Neuronal Function

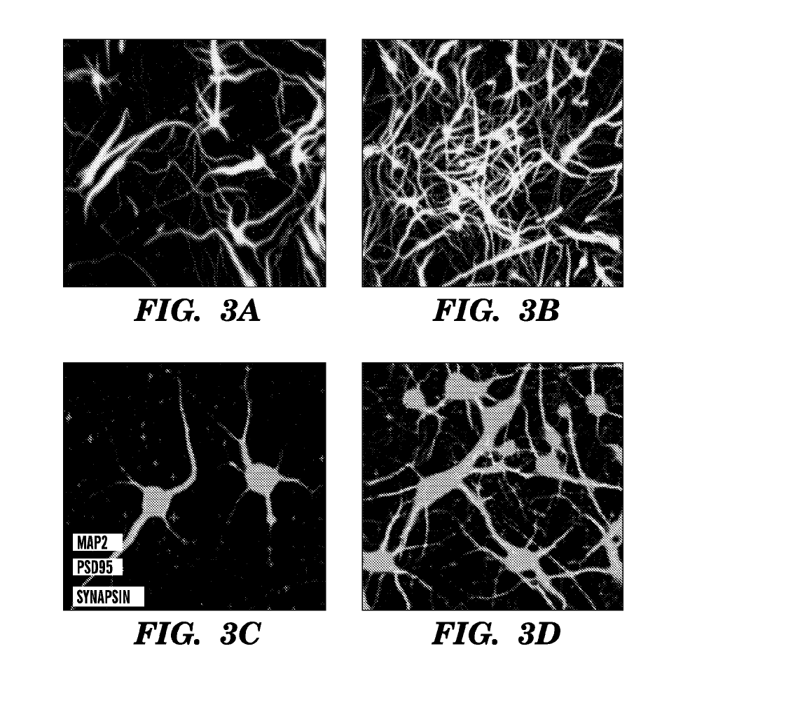

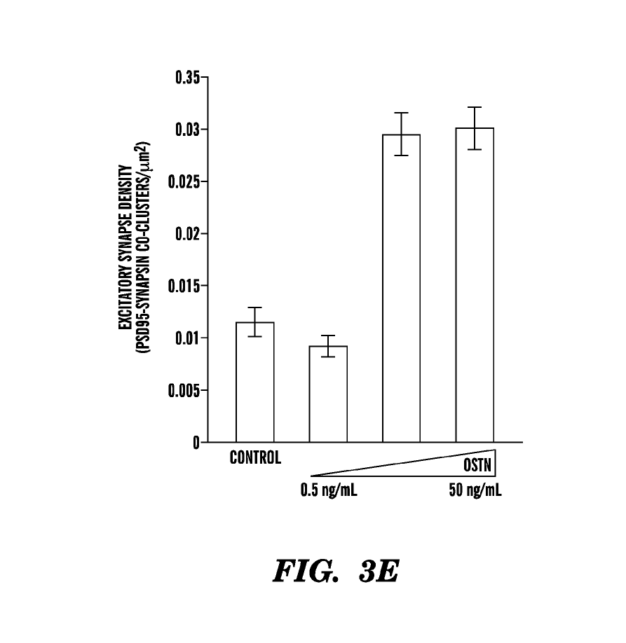

No role has previously been described for OSTN in nervous system development or particularly human nervous system development; however, OSTN has been reported to be secreted from bone-forming cells to promote the proliferation of bone precursors. In order to assess the roles of OSTN in human neuronal function, human primary neuronal cultures were treated with increasing concentrations of an osteocrin-inducing agent, e.g., but not limited to, a recombinant human OSTN protein. After about 6 days, samples were fixed and stained for neuronal markers, e.g., dendritic and/or synaptic markers, to examine effects on neuronal connectivity (FIGS. 3A-3D). Neuronal excitatory synaptic connections can be assayed by any methods known in the art, e.g., by quantifying the density of closely apposed puncta labeling pre- and post-synaptic markers along neuronal dendrites. Using this assay, OSTN-treated cells showed a robust (e.g., at least about 3-fold or higher) increase in excitatory synapse density as compared to mock-treated controls (FIG. 3E). Increases were also observed in dendritic density and total dendritic length following OSTN treatment (data not shown).

Without wishing to be bound by theory, these increases in excitatory synapse density, dendritic density, and/or total dendritic length, in some embodiments, can represent a direct effect of OSTN on synaptic and/or dendritic function. In some embodiments, these increased changes can reflect a more general effect on neuronal health/survival. As osteocrin can promote human neuronal connectivity, delivery of an osteocrin-inducing agent (e.g., but not limited to, a recombinant OSTN or a small molecule OSTN agonist) to neural cells can provide a therapeutic neuro-protective intervention that can be used to, e.g., slow or reverse the neuronal atrophy characteristic of a wide variety of human neurodegenerative conditions, e.g., Alzheimer's disease.

### Example 3

### Effects on an Osteocrin-Inducing Agent on Human Neuronal Survival and Axonal Growth

After the inventors' discovery of OSTN regulating synapse formation in human neuronal cultures, it was next sought to investigate a potential role for OSTN at the earlier stages of neuronal development. It is generally believed that soon after neurons differentiate from neuronal progenitors during early development of the central nervous system, the neurons become susceptible to cell death through intrinsic activation of the programmed cell death (PCD). In fact, approximately half of the immature neurons produced dies through PCD. During this stage, neuronal survival can be promoted through target-derived neurotrophic factors. One of the prime molecules that control the survival and many other aspects of the neuronal development is brain-derived neurotrophic factor (BDNF). BDNF is a secreted molecule and its expression and secretion is highly regulated by neuronal activity.

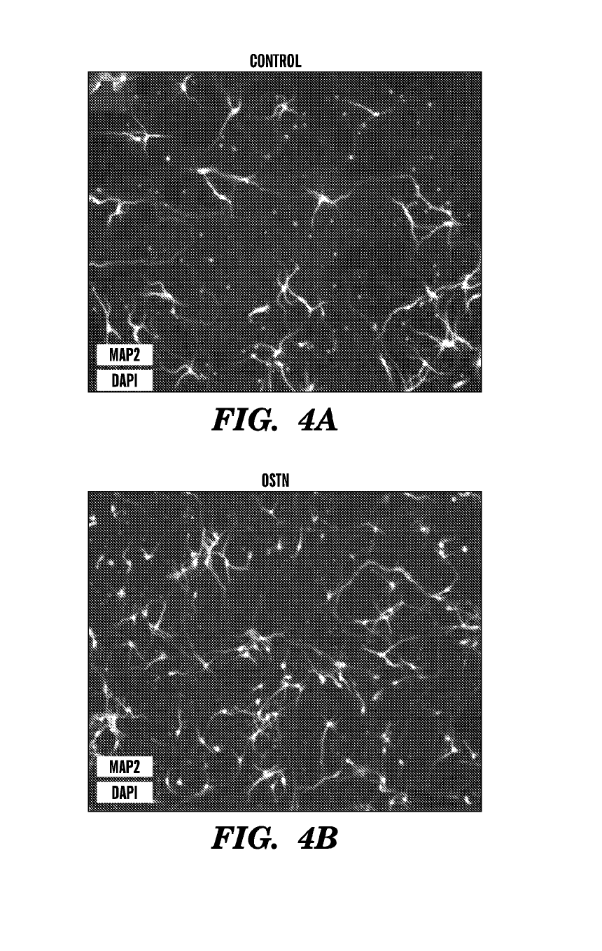

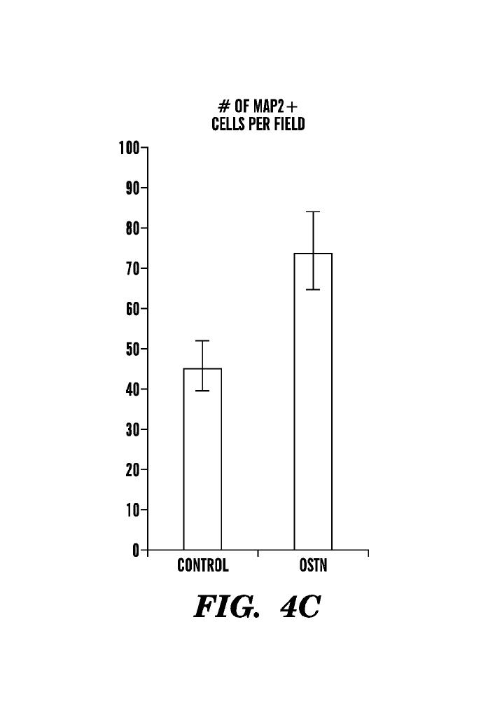

As presented herein, the inventors discovered OSTN as a human-specific activity-regulated secreted molecule. To determine whether OSTN also regulates neuronal survival, e.g., whether OSTN has a “trophic” effect on human neurons, human neurons were cultured at a relatively sparse density, where trophic support from neighboring cells is expected to be minimum. Neurons were grown, e.g., at a density of about 40,000 cells/cm², in the presence or absence of OSTN (e.g., ˜50 ng/ml) for a period of time, e.g., about 6 days between DIV4 to DIV10. After the period of time (e.g., about 6 days), samples were fixed and stained for dendritic and/or nuclear markers (FIGS. 4A-4B). Neuronal survival was assayed, for example, by quantifying the number of MAP2 positive cells after the neurons were cultured with or without OSTN for a pre-determined period of time (e.g., detected at DIV10). Using this assay, OSTN-treated cells showed a robust (˜2-fold) increase in neuronal survival, as compared to mock-treated controls (FIG. 4C). Such significant increase in neuronal survival was not observed when neurons were grown at standard or higher densities (e.g., about 80,000 to about 130,000 cells/cm²) where neighboring neurons could provide trophic support. In some embodiments, the trophic support can include endogenously secreted OSTN (data not shown).

It has been previously showed that neuronal survival and axonal growth are closely linked. One of the neuronal survival requirements is the ability of inducing axon outgrowths of neurons to contact their targets. BDNF and other neurotrophic factors have been previously shown to regulate both neuronal survival and axonal extension. Without wishing to be bound by theory, since human axons can be up to a meter long and once they are generated, they can generally last for decades, human brain may produce human-specific molecules such as OSTN to regulate axonal development.

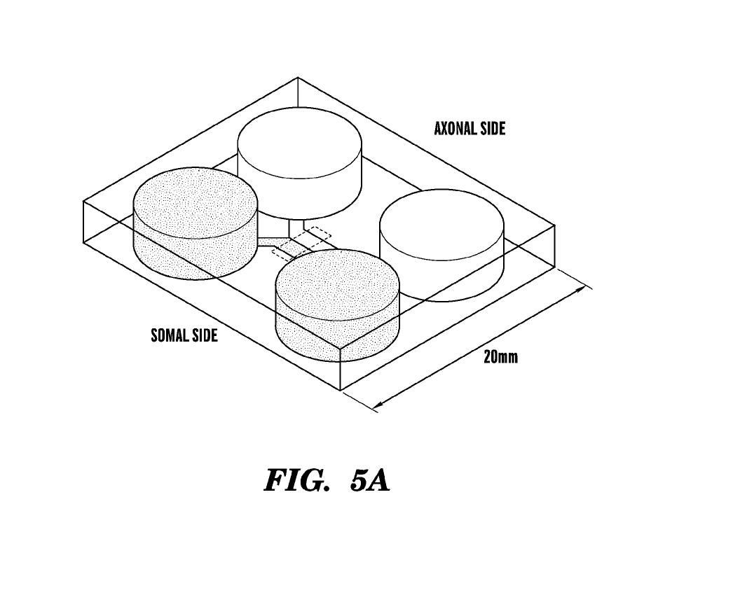

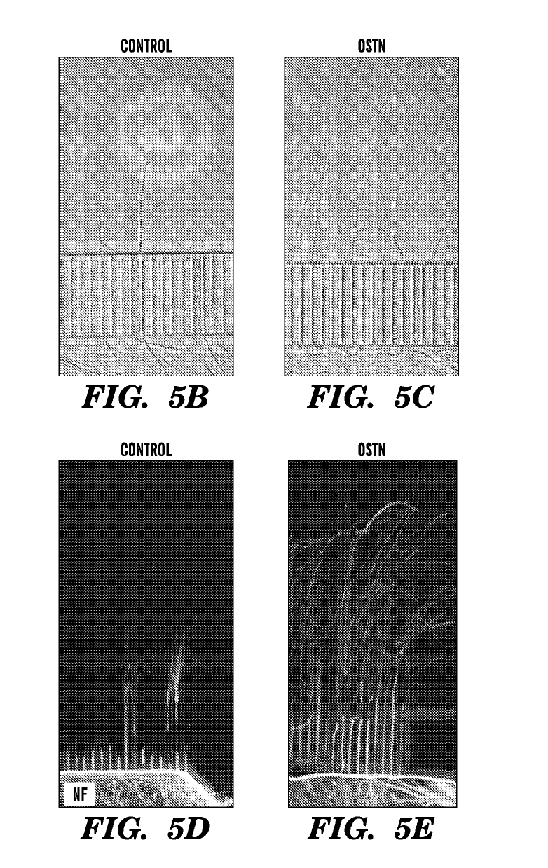

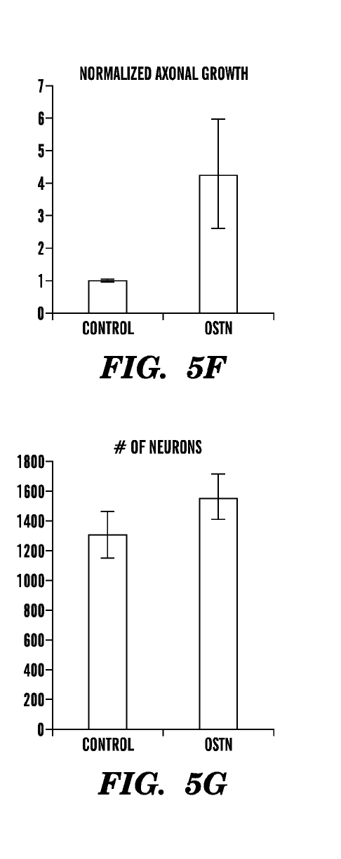

To assess the effect of OSTN on axonal outgrowth, a human axonal growth assay was developed by using microfluidic chambers (e.g., obtained from Xona Microfluidics, Temecula, Calif.) (FIG. 5A). In this assay, primary human neurons were seeded in the presence or absence of OSTN into the microfluidic chambers and cultured for a sufficient period of time to allow their axons grow through microchannels that were designed to be small enough to only allow axons, not dendrites, to pass through. For example, after about 21 days, axons were imaged both live (FIGS. 5B-5C) and after stained with an axonal marker, e.g., an anti-neurofilament antibody (FIGS. 5D-5E). Human axons grew slowly in the absence of OSTN and at DIV21 only few axonal processes were visible (FIGS. 5B and 5D). However, in the presence of OSTN, neurons grow axons of dramatic lengths and complexities (FIGS. 5C and 5E). This significant increase in axonal growth in the presence of OSTN (as compared to the axonal growth in the absence of OSTN) is not contributed by a difference in the number of neuronal cells in the chambers, as the cell number did not appear to change with the OSTN treatment (FIG. 5G). In addition, a dendritic marker (e.g., MAP2) was detected only in the main microfluidic chamber, not in microchannels where axons, but not dendrites, can pass through, indicating that the cellular outgrowths extended into the microchannels were axonal extensions (data not shown). These findings shown that human-specific activity-regulated factor OSTN is a regulator of the axonal growth in human neurons.

Accordingly, in some embodiments, OSTN can regulate neuronal survival. IN some embodiments, OSTN can regulate axonal growth. In some embodiments, OSTN can regulate synapse formation. Since OSTN has been shown herein to promote neuronal survival and/or axonal growth of human neurons, OSTN can be used in therapeutic treatment of a wide spectrum of neurodegenerative diseases as well as diseases and conditions where axonal growth and/or regeneration need to be promoted.

| SEQUENCE LISTING | |
|---|---|
| Homo sapiens osteocrin(OSTN), mRNA(NM_198184.1) | |
| (SEQ ID NO: 1) | |
| 1 | atgctggact ggagattggc aagtgcacat ttcatcctgg ctgtgacact gacactgtgg |
| 61 | agctcaggaa aagtcctctc agtagatgta acaacaacag aggcctttga ttctggagtc |
| 121 | atagatgtgc agtcaacacc cacagtcagg gaagagaaat cagccactga cctgacagca |
| 181 | aaactcttgc ttcttgatga attggtgtcc ctagaaaatg atgtgattga gacaaagaag |
| 241 | aaaaggagtt tctctggttt tgggtctccc cttgacagac tctcagctgg ctctgtagat |
| 301 | cacaaaggta aacagaggaa agtagtagat catccaaaaa ggcgatttgg tatccccatg |
| 361 | gatcggattg gtagaaaccg gctttcaaat tccagaggct aa |
| Homo sapiens osteocrin precursor, protein (NP_937827.1) | |
| (SEQ ID NO: 2) | |
| 1 | mldwrlasah filavtltlw ssgkvlsvdv ttteafdsgv idvqstptvr eeksatdlta |
| 61 | klllldelvs lendvietkk krsfsgfgsp ldrlsagsvd hkgkqrkvvd hpkrrfgipm |
| 121 | drigrnrlsn srg |

It is understood that the foregoing detailed description and examples are illustrative only and are not to be taken as limitations upon the scope of the invention. Various changes and modifications to the disclosed embodiments, which will be apparent to those of skill in the art, may be made without departing from the spirit and scope of the present invention. Further, all patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of increasing neuronal connectivity of a population of neural cells comprising contacting the population of neural cells with a composition comprising an effective amount of an osteocrin-inducing agent.

2.-63. (canceled)

64. The method of claim 1, wherein the increase in neuronal connectivity of the population of neural cells is due to an increase in neuronal survival.

65. The method of claim 1, wherein the osteocrin-inducing agent comprises a recombinant osteocrin protein or a peptidomimetic thereof.

66. The method of claim 1, wherein the osteocrin-inducing agent comprises a recombinant osteocrin-encoding gene.

67. The method of claim 1, wherein the composition further comprises a neural stem cell.

68. The method of claim 1, wherein the osteocrin-inducing agent comprises a small molecule that induces expression of secreted osteocrin.

69. The method of claim 68, wherein the small molecule is a ligand for a natriuretic peptide clearance receptor.

70. The method of claim 68, wherein the small molecule excludes a PPAR-gamma agonist.

71. The method of claim 1, wherein the population of neural cells is present in a cell culture.

72. The method of claim 71, wherein the population of neural cells in the cell culture comprise human neural cells.

73. The method of claim 1, wherein the population of neural cells is present in a subject.

74. The method of claim 73, wherein the subject is diagnosed as having, or having a risk for, cognitive impairment.

75. The method of claim 73, wherein the subject is diagnosed as having, or having a risk for, a neurodegenerative condition.

76. The method of claim 75, wherein the neurodegenerative condition is Alzheimer's disease.

77. The method of claim 73, further comprising selecting a subject diagnosed as having, or having a risk for, cognitive impairment or a neurodegenerative condition prior to the contacting.

78. The method of claim 73, wherein the subject is a human subject.

79. The method of claim 1, wherein the effective amount of the osteocrin-inducing agent is sufficient to increase the neuronal connectivity of the population of neural cells by at least about 10%, as compared to a control population of neural cells not contacted with the osteocrin-inducing agent.

80. An assay for determining a risk for a neurodegenerative disorder in a human subject comprising:
a. subjecting a test sample derived from the brain of a human subject, who is determined to have, or have symptoms of, cognitive impairment, to at least one analysis to determine expression of osteocrin in the test sample;
b. comparing the expression of osteocrin with a reference value using a non-human machine, wherein the reference value corresponds to expression of osteocrin in the brain of a normal healthy subject;
c. identifying the subject to have, or have a risk for, a neurodegenerative disorder when the expression of osteocrin in the test sample is lower than the reference value by at least about 10%; and
d. optionally administering to the subject an osteocrin-inducing agent if the comparison indicates that a subject is diagnosed as having, or having a risk for, a neurodegenerative disorder.

81. The assay of claim 80, wherein said at least one analysis is selected from the group consisting of western blot, enzyme linked absorbance assay, mass spectrometry, immunoassay, flow cytometry, immunohistochemical analysis, PCR reaction, real-time quantitative PCR, and any combinations thereof.

82. The assay of claim 80, wherein the neurodegenerative condition is Alzheimer's disease.


SEQUENCE LISTING (3 sequences)

SEQ ID NO: 1 - DNA, 402 bp, Homo sapiens
atgctggactggagattggcaagtgcacatttcatcctggctgtgacactgacactgtggagctcaggaaaagtcctctcagtagatgtaacaacaacagaggcctttgattctggagtcatagatgtgcagtcaacacccacagtcagggaagagaaatcagccactgacctgacagcaaaactcttgcttcttgatgaattggtgtccctagaaaatgatgtgattgagacaaagaagaaaaggagtttctctggttttgggtctccccttgacagactctcagctggctctgtagatcacaaaggtaaacagaggaaagtagtagatcatccaaaaaggcgatttggtatccccatggatcggattggtagaaaccggctttcaaattccagaggctaa

SEQ ID NO: 2 - protein, 133 aa, Homo sapiens
MLDWRLASAHFILAVTLTLWSSGKVLSVDVTTTEAFDSGVIDVQSTPTVREEKSATDLTAKLLLLDELVSLENDVIETKKKRSFSGFGSPLDRLSAGSVDHKGKQRKVVDHPKRRFGIPMDRIGRNRLSNSRG

SEQ ID NO: 3 - protein, 122 aa, Artificial Sequence
  Description of Artificial Sequence Synthetic polynucleotide
MRGSHHHHHHGMASHMVDVTTTEAFDSGVIDVQSTPTVREEKSATDLTAKLLLLDELVSLENDVIETKKKRSFSGFGSPLDRLSAGSVDHKGKQRKVVDHPKRRFGIPMDRIGRNRLSNSRG